(19) United States
(12) Patent Application Publication
(10) Pub. No.: US 2024/0368684 A1
(43) Pub. Date: Nov. 7, 2024

(54) QUALITY CONTROL FOR REPORTER SCREENING ASSAYS

(71) Applicant: Octant, Inc., Emeryville, CA (US)

(72) Inventors: Carter Barger, Albany, CA (US); Sriram Kosuri, Berkeley, CA (US); Scott Simpkins, Oakland, CA (US); Jeffrey Tang, Daly City, CA (US)

(21) Appl. No.: 18/750,966

(22) Filed: Jun. 21, 2024

Related U.S. Application Data
(63) Continuation of application No. PCT/US2020/053472, filed on Sep. 30, 2020.
(60) Provisional application No. 63/292,841, filed on Dec. 22, 2021.

(51) Int. Cl. C12Q 1/6869; C12N 15/10; C12Q 1/6806

(52) U.S. Cl. CPC C12Q 1/6869; C12N 15/1096; C12Q 1/6806; C12Q 2600/166

(57) ABSTRACT

Described herein is a method of quality assurance for a high-throughput screening system using control nucleic acids to detect and diagnose sample-loss, contamination, and suboptimal process steps.

### CROSS REFERENCE

This application is a continuation of International Application PCT/US2022/053472 filed Dec. 20, 2022 which claims the benefit of U.S. Provisional Application No. 63/292,841, filed Dec. 22, 2021, which are incorporated herein by reference in their entirety.

### BACKGROUND

High throughput screening (HTS) may include multiple steps, which may not perform optimally under certain conditions. Inefficiencies may be introduced due to errors in liquid handling, suboptimal lysis steps, suboptimal enzymatic reactions (including DNase digestion, reverse transcription, amplification, and sequencing), and contamination. Described herein are methods and process steps to control for inefficiencies throughout the high throughput screening process.

### SUMMARY

High throughput screening (HTS) has the potential to reveal much about the biology of cells and accelerate the discovery of new therapeutic molecules. However, HTS as an end-to-end process is beset with uncertainty as the most useful assays (e.g., those that deploy multiple orthogonal readouts) are subject to variability and data loss introduced during sample processing and data acquisition. The methods described herein allow researchers to generate higher confidence data and exclude data of questionable reliability.

Described herein are methods of improving the data output of high-throughput screening assays. The methods are most useful for assays that employ next-generation sequencing as a readout, as such assays have multiple steps, which can introduce multiple sources of variation, contamination, and/or data loss. The methods described herein involve using control nucleic acids added into an end-to-end process at various steps, which allows researchers to identify and exclude unreliable data.

Described herein is a method of quality assurance for a high-throughput screening system the method comprising: (a) providing a first composition comprising a plurality of cells; (b) lysing the plurality of cells to obtain a lysed cell composition, wherein the lysed cell composition comprises a first plurality of control nucleic acids, and a second plurality of control nucleic acids, or a first plurality and a second plurality of control nucleic acids; (c) preparing a reverse transcription reaction comprising at least a portion of the lysed cell composition and a third plurality of control nucleic acids, a fourth plurality of control nucleic acids, or a third plurality and a fourth plurality of control nucleic acids; (d) reverse transcribing the reverse transcription reaction to obtain reverse transcribed nucleic acids; (e) preparing a sequencing library comprising the reverse transcribed nucleic acids; and (f) performing a sequencing reaction on the sequencing library to obtain sequence information for: (i) a plurality of reporters; and (ii) one or more of the first plurality of control nucleic acids, the second plurality of control nucleic acids, the third plurality of control nucleic acids, or the fourth plurality of control nucleic acids. In certain embodiments, the method further comprises contacting the lysed cell composition with a DNA degrading agent. In certain embodiments, the method further comprises contacting the lysed cell composition with a fifth plurality of control nucleic acids before contacting the lysed cell composition with the DNA degrading agent. In certain embodiments, the method further comprises contacting the plurality of cells with a test agent. In certain embodiments, contacting the plurality of cells with the test agent is prior to (b). In certain embodiments, the sequencing information for the plurality of reporters provides information on an mRNA expression level for at least one reporter of the plurality of reporters. In certain embodiments, the at least one reporter comprises a barcode sequence. In certain embodiments, the barcode sequence is operably coupled to a promoter and/or enhancer element. In certain embodiments, the method further comprises performing a sequencing reaction on the sequencing library to obtain sequencing information for (i) a plurality of reporters; and (ii) the first plurality of control nucleic acids, the second plurality of control nucleic acids, and the fourth plurality of control nucleic acids. In certain embodiments, the first plurality of control nucleic acids comprise RNA nucleic acids. In certain embodiments, the first plurality of control nucleic acids is added to the plurality of cells prior to lysis. In certain embodiments, the first plurality of control nucleic acids comprise a first nucleotide sequence, second nucleotide sequence, third nucleotide sequence, and/or fourth nucleotide sequence which are distinguishable from each other. In certain embodiments, the method further comprises normalizing sequence information for at least one reporter of the plurality of reporters to the first plurality of control nucleic acids. In certain embodiments, the second plurality of control nucleic acids comprise double stranded DNA nucleic acids. In certain embodiments, the third plurality of control nucleic acids comprise RNA nucleic acids. In certain embodiments, the method further comprises determining a relative abundance of the second plurality of control nucleic acids amongst the reverse transcribed nucleic acids. In certain embodiments, the fourth plurality of control nucleic acids comprise single-stranded DNA nucleic acids. In certain embodiments, the method further comprises determining a control sequence that is not a sequence from the plurality of reporters the first plurality of control nucleic acids, the second plurality of control nucleic acids, and the fourth plurality of control nucleic acids. In certain embodiments, the plurality of reporters comprise at least 10 reporters, at least 100 reporters, at least 1,000 reporters, at least 5,000 reporters, at least 10,000 reporters. In certain embodiments, each of the at least 10 reporters, at least 100 reporters, at least 1,000 reporters, at least 5,000 reporters, at least 10,000 reporters comprise a unique barcode sequence. In certain embodiments, each of the at least 10 reporters, at least 100 reporters, at least 1,000 reporters, at least 5,000 reporters, at least 10,000 reporters uniquely identify a target molecule. In certain embodiments, the target molecule is a polypeptide expressed by the plurality of cells. In certain embodiments, the sequencing reaction is high throughput sequencing reaction.

### BRIEF DESCRIPTION OF THE DRAWINGS

The novel features described herein are set forth with particularity in the appended claims. A better understanding of the features and advantages of the features described herein will be obtained by reference to the following detailed description that sets forth illustrative examples, in which the principles of the features described herein are utilized, and the accompanying drawings of which:

FIG. 1 shows exemplary next generation sequencing read output from an HTS assay; wells with read numbers below the dotted line were rejected.

FIG. 2 shows a plate map of rejected wells generated using the quality control criteria described herein.

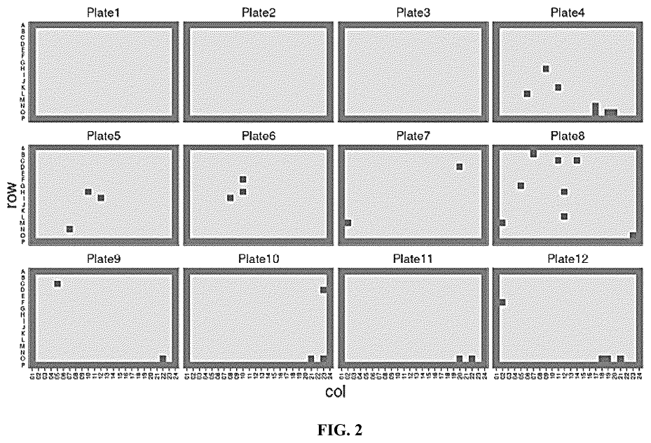

FIG. 3 shows read counts of double stranded DNA control spike-in from samples before and after being treated with DNase.

FIG. 4 shows read counts of single stranded DNA control spike-in from samples before and after being treated with reverse transcriptase.

FIG. 5 shows read counts of various pluralities of control nucleic acids after one or more steps are performed.

FIG. 6 shows an example process with various pluralities of nucleic spikes added.

FIG. 7 shows read counts of various pluralities of nucleic acids.

FIG. 8 shows relevant ratios for analyzing step performance and reliable data for various assays and steps within the assays.

### DETAILED DESCRIPTION

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein the term “about” refers to an amount that is near the stated amount by 10% or less.

The terms “polypeptide” and “protein” are used interchangeably to refer to a polymer of amino acid residues, and are not limited to a minimum length. Polypeptides, including the provided target polypeptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The polypeptides described herein can be encoded by a nucleic acid. A nucleic acid is a type of polynucleotide comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In the expression vectors regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a chromosomal location. Vectors can comprise sequences that direct site-specific integration into a defined location or restricted set of sites in the genome (e.g., AttP-AttB recombination). Additionally, vectors can comprise sequences derived from transposable elements.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). Percent homology of sequences can be determined using the most recent version of BLAST, as of the filing date of this application.

### Assays

Described herein are methods of quality control for high throughput screening assays. Such assays are cell based and comprise a plurality of cells expressing different potential target polypeptides. The activity of a screening library of, for example, small molecules or polypeptides, against a target polypeptide can be determined using various reporter assays that may be activated by the target polypeptide, or the activity of the target polypeptide by downstream mediators. In certain embodiments, the reporter comprises a barcode (e.g., index sequence, also referred to herein as a “unique molecular identifier”) that uniquely maps to a target polypeptide allowing the activity on the polypeptide to be determined by a sequencing assay. In general, a library of cells comprising different target polypeptides may comprise at least 100, 200, 300, 400, 500, 600, 700, 800, 900, 1000 or more different polypeptides uniquely marked by a barcode. The assays of this disclosure can be cared out in 96-, 384, or 1,536 well plates. The reporters may also comprise a visually trackable gene that can generally mark reporter activation (e.g., a fluorescent or luminescent reporter gene). In the assays described herein different cells comprising different target polypeptides (and barcodes) may be present in the same well. In certain embodiments, some or all of a plurality of cells may not express a particular target and may comprise a biological reporter comprising a UMI. Such biological reporters may comprise a promoter, repressor, or enhancer element operatively coupled to the UMI and optionally an additional reporter gene such as a luciferase enzyme or a fluorescent protein.

A non-limiting example of an assay design by the method described herein may comprise: 1) seeding cells in a well of a well plate; 2) contacting the cells with a test agent allowing time for the test agent to affect reporter activation (e.g., barcode expression); 3) lysing the cells to release nucleic acids from the cells, 4) DNase treatment of the lysates to remove contaminating genomic DNA, 5) reverse transcribing and/or amplifying the reverse transcribed mRNA to create cDNA or amplified cDNA (e.g., RT-PCR); 6) preparing the cDNA for a next generation sequencing reaction; and 7) sequencing the prepared cDNA. The steps described above may be performed for each well in the assay. With respect to steps 3) through 7), various nucleic acids are released and/or treated after the cells are seeded in the well and treated with a test agent. With respect to the lysis step, the nucleic acids are released from the cells. Those nucleic acids may be treated with DNase, which cleaves and removes double stranded DNA. The RNA is then reverse transcribed into single stranded DNA through reverse transcriptase, and then further amplified through PCR resulting in double stranded DNA. Thus, through the steps of the assay, multiple nucleic acids are introduced and/or generated, and each of the nucleic acids may be sequenced and identified. By using the control nucleic acids added during the various steps, as well as the read counts, amounts, or ratios of the control nucleic acids detected during each step or, in certain embodiments, at the final sequencing stage by using the control nucleic acid sequences (also referred to herein as various nucleic acid “spike ins” when added to a well of an assay during a process step), the effectiveness of each step can be assessed.

For each step of the non-limiting example described above, certain conditions may affect the effectiveness of each step (e.g., how many cells are lysed during the lysing step, the efficiency of the DNase step, how much cDNA or amplified cDNA is created during the reverse transcription step, etc.) which may affect how many reads for cDNA associated with or converted during that step, thus indicating what conditions lead to better performance or transfers when using assays with different conditions. As described below, the use of control nucleic acids that can be identified throughout the method allow for indications of how effective each step is, and as a result, can lead to optimization of the steps by altering conditions so that the steps are as efficient as possible. These process controls can also increase the ability to normalize and compare data across assays or plates. These process controls can also allow identification of partitions or wells in assays that should be excluded from an analysis.

Additionally, the read counts of the various control nucleic acids during the various steps may indicate whether each step of the process has been effectively performed. For example, if 10,000 nucleic acids (e.g., DNA spike ins) are added to a well during the lysis step, and those 10,000 nucleic acids remain after the DNase step where they were meant to be cleaved, leading to a read count of 10,000 for those nucleic acids, it can be determined that the DNase step was not effective for that particular well, and thus, the data should be excluded. On the other hand, if 10,000 RNA spike ins are added during the RT-PCR where they would be expected to eventually be converted to DNA, and a read count of 10,000 reads of the DNA is counted at the end of the RT-PCR step when an expected read count would be 5,000 reads, it can be determined that at least one step of the assay or measurement of the assay was either incorrect or ineffective since the actual read count was so much higher than the expected read count, and thus, the data from that well should be excluded.

### Control Nucleic Acids

Control nucleic acids can be added during one or more of a lysis step, a DNase step, a reverse transcriptase reaction, an RT-PCR reaction, or a next generation sequencing library preparation reaction. The control nucleic acids comprise a known sequence that differ amongst the different types of control nucleic acids, allowing for identification of variability at different process steps.

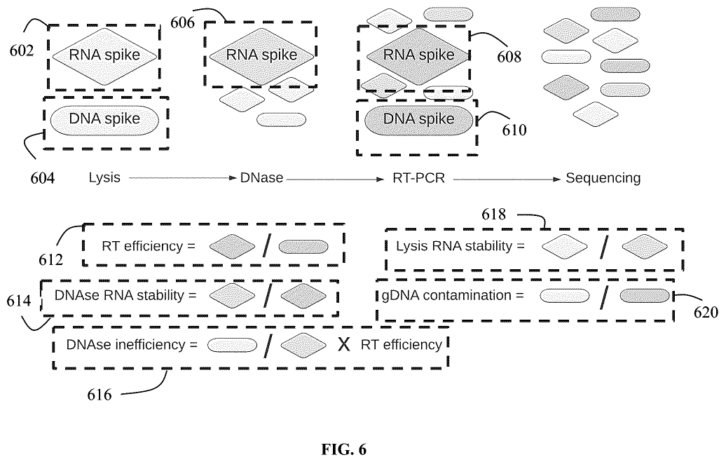

FIG. 6 depicts an example process where control nucleic acids are added during various steps, such as the Lysis step, DNase step, and RT-PCR step, as described above. When added to a well during a step of the assay, the control nucleic acids may be referred to as “spike ins” of a various nucleic acid (e.g., DNA or RNA). Comparing the known amount of control nucleic acids to the amount of reads for those nucleic acids sequenced later on gives insight to the effectiveness of each step. The amount of control nucleic acids added at each step may vary. Additionally, the amount of control nucleic acids may be limited to within a range (e.g., from 1,000 to 10,000 control nucleic acids in a plurality of nucleic acids) in order to not interfere with the effectiveness of each step. For example, if the amount of control nucleic acids added during a step is too low, there will not be enough control nucleic acids added to calibrate a step (e.g., change one or more conditions of the step) or obtain a preferred read count (e.g., 1,000 reads per well). On the other hand, if the amount of control nucleic acids added is too high, it will interfere with observable performance of the step (e.g., as there may be too many of nucleic acid spike ins, which use too much of the available reagents to get reads from UMIs generated by reporters), and thus, also will influence the read counts.

Control nucleic acids can be added to samples at various process steps based on an absolute number (or a concentration equivalent to an absolute number). In some embodiments, the amount of control nucleic acids (e.g., an RNA or DNA spike-in) added at a particular process step comprises about 1,000 nucleic acids to about 50,000 nucleic acids. In some embodiments, the amount of nucleic acids (e.g., an RNA or DNA spike in) comprises about 1,000 nucleic acids to about 2,000 nucleic acids, about 1,000 nucleic acids to about 3,000 nucleic acids, about 1,000 nucleic acids to about 4,000 nucleic acids, about 1,000 nucleic acids to about 5,000 nucleic acids, about 1,000 nucleic acids to about 7,500 nucleic acids, about 1,000 nucleic acids to about 10,000 nucleic acids, about 1,000 nucleic acids to about 12,500 nucleic acids, about 1,000 nucleic acids to about 15,000 nucleic acids, about 1,000 nucleic acids to about 20,000 nucleic acids, about 1,000 nucleic acids to about 30,000 nucleic acids, about 1,000 nucleic acids to about 50,000 nucleic acids, about 2,000 nucleic acids to about 3,000 nucleic acids, about 2,000 nucleic acids to about 4,000 nucleic acids, about 2,000 nucleic acids to about 5,000 nucleic acids, about 2,000 nucleic acids to about 7,500 nucleic acids, about 2,000 nucleic acids to about 10,000 nucleic acids, about 2,000 nucleic acids to about 12,500 nucleic acids, about 2,000 nucleic acids to about 15,000 nucleic acids, about 2,000 nucleic acids to about 20,000 nucleic acids, about 2,000 nucleic acids to about 30,000 nucleic acids, about 2,000 nucleic acids to about 50,000 nucleic acids, about 3,000 nucleic acids to about 4,000 nucleic acids, about 3,000 nucleic acids to about 5,000 nucleic acids, about 3,000 nucleic acids to about 7,500 nucleic acids, about 3,000 nucleic acids to about 10,000 nucleic acids, about 3,000 nucleic acids to about 12,500 nucleic acids, about 3,000 nucleic acids to about 15,000 nucleic acids, about 3,000 nucleic acids to about 20,000 nucleic acids, about 3,000 nucleic acids to about 30,000 nucleic acids, about 3,000 nucleic acids to about 50,000 nucleic acids, about 4,000 nucleic acids to about 5,000 nucleic acids, about 4,000 nucleic acids to about 7,500 nucleic acids, about 4,000 nucleic acids to about 10,000 nucleic acids, about 4,000 nucleic acids to about 12,500 nucleic acids, about 4,000 nucleic acids to about 15,000 nucleic acids, about 4,000 nucleic acids to about 20,000 nucleic acids, about 4,000 nucleic acids to about 30,000 nucleic acids, about 4,000 nucleic acids to about 50,000 nucleic acids, about 5,000 nucleic acids to about 7,500 nucleic acids, about 5,000 nucleic acids to about 10,000 nucleic acids, about 5,000 nucleic acids to about 12,500 nucleic acids, about 5,000 nucleic acids to about 15,000 nucleic acids, about 5,000 nucleic acids to about 20,000 nucleic acids, about 5,000 nucleic acids to about 30,000 nucleic acids, about 5,000 nucleic acids to about 50,000 nucleic acids, about 7,500 nucleic acids to about 10,000 nucleic acids, about 7,500 nucleic acids to about 12,500 nucleic acids, about 7,500 nucleic acids to about 15,000 nucleic acids, about 7,500 nucleic acids to about 20,000 nucleic acids, about 7,500 nucleic acids to about 30,000 nucleic acids, about 7,500 nucleic acids to about 50,000 nucleic acids, about 10,000 nucleic acids to about 12,500 nucleic acids, about 10,000 nucleic acids to about 15,000 nucleic acids, about 10,000 nucleic acids to about 20,000 nucleic acids, about 10,000 nucleic acids to about 30,000 nucleic acids, about 10,000 nucleic acids to about 50,000 nucleic acids, about 12,500 nucleic acids to about 15,000 nucleic acids, about 12,500 nucleic acids to about 20,000 nucleic acids, about 12,500 nucleic acids to about 30,000 nucleic acids, about 12,500 nucleic acids to about 50,000 nucleic acids, about 15,000 nucleic acids to about 20,000 nucleic acids, about 15,000 nucleic acids to about 30,000 nucleic acids, about 15,000 nucleic acids to about 50,000 nucleic acids, about 20,000 nucleic acids to about 30,000 nucleic acids, about 20,000 nucleic acids to about 50,000 nucleic acids, or about 30,000 nucleic acids to about 50,000 nucleic acids. In some embodiments, the amount of nucleic acids (e.g., an RNA or DNA spike in) comprises about 1,000 nucleic acids, about 2,000 nucleic acids, about 3,000 nucleic acids, about 4,000 nucleic acids, about 5,000 nucleic acids, about 7,500 nucleic acids, about 10,000 nucleic acids, about 12,500 nucleic acids, about 15,000 nucleic acids, about 20,000 nucleic acids, about 30,000 nucleic acids, or about 50,000 nucleic acids. In some embodiments, the amount of nucleic acids (e.g., an RNA or DNA spike in) comprises at least about 1,000 nucleic acids, about 2,000 nucleic acids, about 3,000 nucleic acids, about 4,000 nucleic acids, about 5,000 nucleic acids, about 7,500 nucleic acids, about 10,000 nucleic acids, about 12,500 nucleic acids, about 15,000 nucleic acids, about 20,000 nucleic acids, or about 30,000 nucleic acids. In some embodiments, the amount of nucleic acids (e.g., an RNA or DNA spike in) comprises at most about 2,000 nucleic acids, about 3,000 nucleic acids, about 4,000 nucleic acids, about 5,000 nucleic acids, about 7,500 nucleic acids, about 10,000 nucleic acids, about 12,500 nucleic acids, about 15,000 nucleic acids, about 20,000 nucleic acids, about 30,000 nucleic acids, or about 50,000 nucleic acids. These spike ins can comprise dsDNA, ssDNA, ssRNA, or a combination thereof. An amount of nucleic acids (e.g., read count/absolute number) may be used to determine the concentration of nucleic acids in a well, and a concentration of nucleic acids in a well may also be used to determine the amount of nucleic acids in the well. In some embodiments, a known amount of nucleic acids may be added to a well at the beginning of a method or step, while a concentration of nucleic acids may be determined after the method or step have been performed.

In some embodiments, the control nucleic acids added to various steps in the assay result in a certain total number of reads for all of the different pluralities of control nucleic acids the expected read count per well in an assay is about 1,000 reads of a control nucleic acid. In some embodiments, the read count per well in an assay is about 0 control nucleic acids to about 150 control nucleic acids. In some embodiments, the read count per well in an assay is about 0 control nucleic acids to about 5 control nucleic acids, about 0 control nucleic acids to about 10 control nucleic acids, about 0 control nucleic acids to about 15 control nucleic acids, about 0 control nucleic acids to about 20 control nucleic acids, about 0 control nucleic acids to about 25 control nucleic acids, about 0 control nucleic acids to about 30 control nucleic acids, about 0 control nucleic acids to about 40 control nucleic acids, about 0 control nucleic acids to about 50 control nucleic acids, about 0 control nucleic acids to about 75 control nucleic acids, about 0 control nucleic acids to about 100 control nucleic acids, about 0 control nucleic acids to about 150 control nucleic acids, about 5 control nucleic acids to about 10 control nucleic acids, about 5 control nucleic acids to about 15 control nucleic acids, about 5 control nucleic acids to about 20 control nucleic acids, about 5 control nucleic acids to about 25 control nucleic acids, about 5 control nucleic acids to about 30 control nucleic acids, about 5 control nucleic acids to about 40 control nucleic acids, about 5 control nucleic acids to about 50 control nucleic acids, about 5 control nucleic acids to about 75 control nucleic acids, about 5 control nucleic acids to about 100 control nucleic acids, about 5 control nucleic acids to about 150 control nucleic acids, about 10 control nucleic acids to about 15 control nucleic acids, about 10 control nucleic acids to about 20 control nucleic acids, about 10 control nucleic acids to about 25 control nucleic acids, about 10 control nucleic acids to about 30 control nucleic acids, about 10 control nucleic acids to about 40 control nucleic acids, about 10 control nucleic acids to about 50 control nucleic acids, about 10 control nucleic acids to about 75 control nucleic acids, about 10 control nucleic acids to about 100 control nucleic acids, about 10 control nucleic acids to about 150 control nucleic acids, about 15 control nucleic acids to about 20 control nucleic acids, about 15 control nucleic acids to about 25 control nucleic acids, about 15 control nucleic acids to about 30 control nucleic acids, about 15 control nucleic acids to about 40 control nucleic acids, about 15 control nucleic acids to about 50 control nucleic acids, about 15 control nucleic acids to about 75 control nucleic acids, about 15 control nucleic acids to about 100 control nucleic acids, about 15 control nucleic acids to about 150 control nucleic acids, about 20 control nucleic acids to about 25 control nucleic acids, about 20 control nucleic acids to about 30 control nucleic acids, about 20 control nucleic acids to about 40 control nucleic acids, about 20 control nucleic acids to about 50 control nucleic acids, about 20 control nucleic acids to about 75 control nucleic acids, about 20 control nucleic acids to about 100 control nucleic acids, about 20 control nucleic acids to about 150 control nucleic acids, about 25 control nucleic acids to about 30 control nucleic acids, about 25 control nucleic acids to about 40 control nucleic acids, about 25 control nucleic acids to about 50 control nucleic acids, about 25 control nucleic acids to about 75 control nucleic acids, about 25 control nucleic acids to about 100 control nucleic acids, about 25 control nucleic acids to about 150 control nucleic acids, about 30 control nucleic acids to about 40 control nucleic acids, about 30 control nucleic acids to about 50 control nucleic acids, about 30 control nucleic acids to about 75 control nucleic acids, about 30 control nucleic acids to about 100 control nucleic acids, about 30 control nucleic acids to about 150 control nucleic acids, about 40 control nucleic acids to about 50 control nucleic acids, about 40 control nucleic acids to about 75 control nucleic acids, about 40 control nucleic acids to about 100 control nucleic acids, about 40 control nucleic acids to about 150 control nucleic acids, about 50 control nucleic acids to about 75 control nucleic acids, about 50 control nucleic acids to about 100 control nucleic acids, about 50 control nucleic acids to about 150 control nucleic acids, about 75 control nucleic acids to about 100 control nucleic acids, about 75 control nucleic acids to about 150 control nucleic acids, or about 100 control nucleic acids to about 150 control nucleic acids. In some embodiments, the read count per well in an assay is about 0 control nucleic acids, about 5 control nucleic acids, about 10 control nucleic acids, about 15 control nucleic acids, about 20 control nucleic acids, about 25 control nucleic acids, about 30 control nucleic acids, about 40 control nucleic acids, about 50 control nucleic acids, about 75 control nucleic acids, about 100 control nucleic acids, or about 150 control nucleic acids. In some embodiments, the read count per well in an assay is at least about 0 control nucleic acids, about 5 control nucleic acids, about 10 control nucleic acids, about 15 control nucleic acids, about 20 control nucleic acids, about 25 control nucleic acids, about 30 control nucleic acids, about 40 control nucleic acids, about 50 control nucleic acids, about 75 control nucleic acids, or about 100 control nucleic acids. In some embodiments, the read count per well in an assay is at most about 5 control nucleic acids, about 10 control nucleic acids, about 15 control nucleic acids, about 20 control nucleic acids, about 25 control nucleic acids, about 30 control nucleic acids, about 40 control nucleic acids, about 50 control nucleic acids, about 75 control nucleic acids, about 100 control nucleic acids, or about 150 control nucleic acids. In some embodiments, the read count per well in an assay is about 100 control nucleic acids to about 1,500 control nucleic acids. In some embodiments, the read count per well in an assay is about 100 control nucleic acids to about 200 control nucleic acids, about 100 control nucleic acids to about 300 control nucleic acids, about 100 control nucleic acids to about 400 control nucleic acids, about 100 control nucleic acids to about 500 control nucleic acids, about 100 control nucleic acids to about 600 control nucleic acids, about 100 control nucleic acids to about 700 control nucleic acids, about 100 control nucleic acids to about 800 control nucleic acids, about 100 control nucleic acids to about 900 control nucleic acids, about 100 control nucleic acids to about 1,000 control nucleic acids, about 100 control nucleic acids to about 1,250 control nucleic acids, about 100 control nucleic acids to about 1,500 control nucleic acids, about 200 control nucleic acids to about 300 control nucleic acids, about 200 control nucleic acids to about 400 control nucleic acids, about 200 control nucleic acids to about 500 control nucleic acids, about 200 control nucleic acids to about 600 control nucleic acids, about 200 control nucleic acids to about 700 control nucleic acids, about 200 control nucleic acids to about 800 control nucleic acids, about 200 control nucleic acids to about 900 control nucleic acids, about 200 control nucleic acids to about 1,000 control nucleic acids, about 200 control nucleic acids to about 1,250 control nucleic acids, about 200 control nucleic acids to about 1,500 control nucleic acids, about 300 control nucleic acids to about 400 control nucleic acids, about 300 control nucleic acids to about 500 control nucleic acids, about 300 control nucleic acids to about 600 control nucleic acids, about 300 control nucleic acids to about 700 control nucleic acids, about 300 control nucleic acids to about 800 control nucleic acids, about 300 control nucleic acids to about 900 control nucleic acids, about 300 control nucleic acids to about 1,000 control nucleic acids, about 300 control nucleic acids to about 1,250 control nucleic acids, about 300 control nucleic acids to about 1,500 control nucleic acids, about 400 control nucleic acids to about 500 control nucleic acids, about 400 control nucleic acids to about 600 control nucleic acids, about 400 control nucleic acids to about 700 control nucleic acids, about 400 control nucleic acids to about 800 control nucleic acids, about 400 control nucleic acids to about 900 control nucleic acids, about 400 control nucleic acids to about 1,000 control nucleic acids, about 400 control nucleic acids to about 1,250 control nucleic acids, about 400 control nucleic acids to about 1,500 control nucleic acids, about 500 control nucleic acids to about 600 control nucleic acids, about 500 control nucleic acids to about 700 control nucleic acids, about 500 control nucleic acids to about 800 control nucleic acids, about 500 control nucleic acids to about 900 control nucleic acids, about 500 control nucleic acids to about 1,000 control nucleic acids, about 500 control nucleic acids to about 1,250 control nucleic acids, about 500 control nucleic acids to about 1,500 control nucleic acids, about 600 control nucleic acids to about 700 control nucleic acids, about 600 control nucleic acids to about 800 control nucleic acids, about 600 control nucleic acids to about 900 control nucleic acids, about 600 control nucleic acids to about 1,000 control nucleic acids, about 600 control nucleic acids to about 1,250 control nucleic acids, about 600 control nucleic acids to about 1,500 control nucleic acids, about 700 control nucleic acids to about 800 control nucleic acids, about 700 control nucleic acids to about 900 control nucleic acids, about 700 control nucleic acids to about 1,000 control nucleic acids, about 700 control nucleic acids to about 1,250 control nucleic acids, about 700 control nucleic acids to about 1,500 control nucleic acids, about 800 control nucleic acids to about 900 control nucleic acids, about 800 control nucleic acids to about 1,000 control nucleic acids, about 800 control nucleic acids to about 1,250 control nucleic acids, about 800 control nucleic acids to about 1,500 control nucleic acids, about 900 control nucleic acids to about 1,000 control nucleic acids, about 900 control nucleic acids to about 1,250 control nucleic acids, about 900 control nucleic acids to about 1,500 control nucleic acids, about 1,000 control nucleic acids to about 1,250 control nucleic acids, about 1,000 control nucleic acids to about 1,500 control nucleic acids, or about 1,250 control nucleic acids to about 1,500 control nucleic acids. In some embodiments, the read count per well in an assay is about 100 control nucleic acids, about 200 control nucleic acids, about 300 control nucleic acids, about 400 control nucleic acids, about 500 control nucleic acids, about 600 control nucleic acids, about 700 control nucleic acids, about 800 control nucleic acids, about 900 control nucleic acids, about 1,000 control nucleic acids, about 1,250 control nucleic acids, or about 1,500 control nucleic acids. In some embodiments, the read count per well in an assay is at least about 100 control nucleic acids, about 200 control nucleic acids, about 300 control nucleic acids, about 400 control nucleic acids, about 500 control nucleic acids, about 600 control nucleic acids, about 700 control nucleic acids, about 800 control nucleic acids, about 900 control nucleic acids, about 1,000 control nucleic acids, or about 1,250 control nucleic acids. In some embodiments, the read count per well in an assay is at most about 200 control nucleic acids, about 300 control nucleic acids, about 400 control nucleic acids, about 500 control nucleic acids, about 600 control nucleic acids, about 700 control nucleic acids, about 800 control nucleic acids, about 900 control nucleic acids, about 1,000 control nucleic acids, about 1,250 control nucleic acids, or about 1,500 control nucleic acids. In some embodiments, the read count per well in an assay is about 500 control nucleic acids to about 15,000 control nucleic acids. In some embodiments, the read count per well in an assay is about 500 control nucleic acids to about 750 control nucleic acids, about 500 control nucleic acids to about 1,000 control nucleic acids, about 500 control nucleic acids to about 1,250 control nucleic acids, about 500 control nucleic acids to about 1,500 control nucleic acids, about 500 control nucleic acids to about 2,000 control nucleic acids, about 500 control nucleic acids to about 3,000 control nucleic acids, about 500 control nucleic acids to about 4,000 control nucleic acids, about 500 control nucleic acids to about 5,000 control nucleic acids, about 500 control nucleic acids to about 7,500 control nucleic acids, about 500 control nucleic acids to about 10,000 control nucleic acids, about 500 control nucleic acids to about 15,000 control nucleic acids, about 750 control nucleic acids to about 1,000 control nucleic acids, about 750 control nucleic acids to about 1,250 control nucleic acids, about 750 control nucleic acids to about 1,500 control nucleic acids, about 750 control nucleic acids to about 2,000 control nucleic acids, about 750 control nucleic acids to about 3,000 control nucleic acids, about 750 control nucleic acids to about 4,000 control nucleic acids, about 750 control nucleic acids to about 5,000 control nucleic acids, about 750 control nucleic acids to about 7,500 control nucleic acids, about 750 control nucleic acids to about 10,000 control nucleic acids, about 750 control nucleic acids to about 15,000 control nucleic acids, about 1,000 control nucleic acids to about 1,250 control nucleic acids, about 1,000 control nucleic acids to about 1,500 control nucleic acids, about 1,000 control nucleic acids to about 2,000 control nucleic acids, about 1,000 control nucleic acids to about 3,000 control nucleic acids, about 1,000 control nucleic acids to about 4,000 control nucleic acids, about 1,000 control nucleic acids to about 5,000 control nucleic acids, about 1,000 control nucleic acids to about 7,500 control nucleic acids, about 1,000 control nucleic acids to about 10,000 control nucleic acids, about 1,000 control nucleic acids to about 15,000 control nucleic acids, about 1,250 control nucleic acids to about 1,500 control nucleic acids, about 1,250 control nucleic acids to about 2,000 control nucleic acids, about 1,250 control nucleic acids to about 3,000 control nucleic acids, about 1,250 control nucleic acids to about 4,000 control nucleic acids, about 1,250 control nucleic acids to about 5,000 control nucleic acids, about 1,250 control nucleic acids to about 7,500 control nucleic acids, about 1,250 control nucleic acids to about 10,000 control nucleic acids, about 1,250 control nucleic acids to about 15,000 control nucleic acids, about 1,500 control nucleic acids to about 2,000 control nucleic acids, about 1,500 control nucleic acids to about 3,000 control nucleic acids, about 1,500 control nucleic acids to about 4,000 control nucleic acids, about 1,500 control nucleic acids to about 5,000 control nucleic acids, about 1,500 control nucleic acids to about 7,500 control nucleic acids, about 1,500 control nucleic acids to about 10,000 control nucleic acids, about 1,500 control nucleic acids to about 15,000 control nucleic acids, about 2,000 control nucleic acids to about 3,000 control nucleic acids, about 2,000 control nucleic acids to about 4,000 control nucleic acids, about 2,000 control nucleic acids to about 5,000 control nucleic acids, about 2,000 control nucleic acids to about 7,500 control nucleic acids, about 2,000 control nucleic acids to about 10,000 control nucleic acids, about 2,000 control nucleic acids to about 15,000 control nucleic acids, about 3,000 control nucleic acids to about 4,000 control nucleic acids, about 3,000 control nucleic acids to about 5,000 control nucleic acids, about 3,000 control nucleic acids to about 7,500 control nucleic acids, about 3,000 control nucleic acids to about 10,000 control nucleic acids, about 3,000 control nucleic acids to about 15,000 control nucleic acids, about 4,000 control nucleic acids to about 5,000 control nucleic acids, about 4,000 control nucleic acids to about 7,500 control nucleic acids, about 4,000 control nucleic acids to about 10,000 control nucleic acids, about 4,000 control nucleic acids to about 15,000 control nucleic acids, about 5,000 control nucleic acids to about 7,500 control nucleic acids, about 5,000 control nucleic acids to about 10,000 control nucleic acids, about 5,000 control nucleic acids to about 15,000 control nucleic acids, about 7,500 control nucleic acids to about 10,000 control nucleic acids, about 7,500 control nucleic acids to about 15,000 control nucleic acids, or about 10,000 control nucleic acids to about 15,000 control nucleic acids. In some embodiments, the read count per well in an assay is about 500 control nucleic acids, about 750 control nucleic acids, about 1,000 control nucleic acids, about 1,250 control nucleic acids, about 1,500 control nucleic acids, about 2,000 control nucleic acids, about 3,000 control nucleic acids, about 4,000 control nucleic acids, about 5,000 control nucleic acids, about 7,500 control nucleic acids, about 10,000 control nucleic acids, or about 15,000 control nucleic acids. In some embodiments, the read count per well in an assay is at least about 500 control nucleic acids, about 750 control nucleic acids, about 1,000 control nucleic acids, about 1,250 control nucleic acids, about 1,500 control nucleic acids, about 2,000 control nucleic acids, about 3,000 control nucleic acids, about 4,000 control nucleic acids, about 5,000 control nucleic acids, about 7,500 control nucleic acids, or about 10,000 control nucleic acids. In some embodiments, the read count per well in an assay is at most about 750 control nucleic acids, about 1,000 control nucleic acids, about 1,250 control nucleic acids, about 1,500 control nucleic acids, about 2,000 control nucleic acids, about 3,000 control nucleic acids, about 4,000 control nucleic acids, about 5,000 control nucleic acids, about 7,500 control nucleic acids, about 10,000 control nucleic acids, or about 15,000 control nucleic acids. While certain read counts are described above, the read counts are exemplary, and other read counts may be determined. Additionally, various control nucleic acids may have different read counts determined in a well. Further, not all nucleic acids need to have the same read counts determined between wells.

Adding control nucleic acids (or pluralities of control nucleic acids) provides a way to determine the effectiveness of one or more steps. For example, the control nucleic acids may be introduced in a step in a known amount, and the reads for those control nucleic acids may be counted at a later time during the step they are introduced or may be counted during a later step. Thus, the read counts of the control nucleic acids during a certain step, in comparison to the amount in which the control nucleic acids were introduced, may provide insight as to how well a step is working (e.g., how efficient the reverse transcriptase reaction is). If a step is determined to be working effectively (e.g., the reads of the control nucleic acid are near an expected amount of reads), then the conditions of the step may not be changed, or may be changed to further increase the effectiveness of the step, while if a step is determined to not be working well (e.g., the reads of the control nucleic acid are not near an expected amount of reads), the conditions of the step may be changed in order to increase effectiveness.

The lysis step may include disrupting the cell membrane in such a way to release nucleic acids from inside the cell. The nucleic acids may include DNA, RNA, or other nucleic acids.

A first plurality of control nucleic acids (e.g., RNA spike 602) can be added during or before the lysis step. In certain embodiments, the first plurality of control nucleic acids may be comprised in a lysing agent or buffer added to cells during cell lysis. In some embodiments, the first plurality of nucleic acids are in a plurality of cells that are seeded in a well, where the cells are then lysed to release internal contents of the cell. In certain embodiments, these nucleic acids comprise RNA. In certain embodiments, these nucleic acids comprise DNA. In certain embodiments, one of the first plurality of control nucleic acids are DNA and the other of the first plurality of control nucleic acids are RNA. This first plurality of nucleic acids can be used (1) to normalize end read counts against a known reference standard, and/or (2) determine ratios of the read counts of the samples of interest to the other control nucleic acids spike-ins can be used as a quantitative measure of the effectiveness of the cell lysis reaction. The first plurality of nucleic acids may be used to normalize end read counts against a known reference standard by comparing the read counts of the first plurality of nucleic acids during one or more later steps (e.g., the sequencing step) to the amount of the first plurality of nucleic acids originally included. The first plurality of nucleic acids may also be used to determine a cell lysis reaction ratio of the read counts by comparing the number of reads of the first plurality of nucleic acids counted during the sequencing step to the number of the first plurality of nucleic acids added to the lysis step. If the cell lysis reaction is ineffective, the cell lysis reaction ratio will be lower, whereas if the cell lysis reaction is effective, the cell lysis reaction will be higher. For example, a cell lysis reaction ratio of 0.1 will indicate that the cell lysis reaction was ineffective, as only 10% of the first plurality of nucleic acids were lysed. By contrast, a cell lysis reaction ratio of 0.95 will indicate that the cell lysis reaction was effective, as 95% of the first plurality of nucleic acids were lysed. Conditions of the lysis step may be altered in order to improve the cell lysis reaction ratio, such as the pH of the assay, the duration of the assay, the amount of cells added, or other conditions.

Other ratios, such as a Lysis RNA stability ratio (e.g., “Lysis RNA stability” 618), may be determined based on the another plurality of nucleic acids added during the lysis step. For example, the Lysis RNA stability ratio may measure the effectiveness of the transfer from the lysis step to the DNase step, and/or determine whether the presence of RNase impacted any of the subsequent steps before and including the RT step. Additionally, the Lysis RNA stability ratio may indicate if the presence of DNase in a well is too strong. For example, if too much DNase is present during the DNase step, the DNase may not only cleave DNA, but may also cleave RNA, which will result in a lower concentration of RNA, and, as a result, lower the Lysis RNA stability ratio. Thus, the Lysis RNA stability ratio may indicate that too much DNase is used during the DNase step. For example, if a read count of the another plurality of nucleic acids (e.g., another RNA spike) is performed at the end of the lysis step, and then another read count of the another plurality of nucleic acids is performed at the beginning of the DNase step, the effectiveness of the transfer may be evaluated using the Lysis RNA stability ratio. Thus, if a Lysis RNA stability ratio of 0.94 indicates that the transfer was effective. By contrast, a Lysis RNA stability ratio of 0.25 indicates that the transfer was ineffective and that certain conditions (e.g., speed, duration, temperature, and/or pH) of the transfer may need to be changed.

A second plurality of control nucleic acids (e.g., DNA spike 604) can be added during or before the lysis step. In some embodiments, the second plurality of nucleic acids are in a plurality of cells that are added during the lysis step, where the cells are then lysed. In certain embodiments, the second plurality of control nucleic acids may be comprised in a lysing agent or buffer added to cells during cell lysis. In certain embodiments, these nucleic acids comprise double-stranded DNA. These double stranded DNAs can be used to determine the effectiveness of a DNase step, thus identifying potential sources of contamination from incompletely DNased samples. For example, read counts of the second plurality of nucleic acids may indicate a DNase inefficiency ratio (e.g., “DNase inefficiency” 616)). The DNase inefficiency ratio may be determined by comparing the read count of RNA related to the second plurality nucleic acids measured during the DNase step to the read count of RNA related to the second plurality of nucleic acids measured during the reverse transcriptase step. In particular, all of or a portion of the second plurality of nucleic acids that are added during the lysis step may be cleaved during the DNase step, resulting in resultant RNA that is counted during the DNase step. Thus, by comparing the known amount of the second plurality of nucleic acids that are added during the lysis step to the read count of RNA during the DNase step, the DNase inefficiency ratio can be determined. For example, a DNase inefficiency ratio of 1,000:10,000 will indicate that 90% of the second plurality of nucleic acids were cleaved during the DNase step. By contrast, a DNase inefficiency ratio of 5,000:10,000 will indicate that only 50% of the second plurality of nucleic acids were cleaved during the DNase step. Conditions of the lysis step may be altered in order to improve the DNase inefficiency ratio, such as the pH of the assay, the duration of the assay, the amount of cells added, or other conditions.

A third plurality of nucleic acids (e.g., RNA spike 608) can be added during the RT-PCR step. In certain embodiments, these nucleic acids comprise RNA. This plurality of nucleic acids can be used to identify failure of or suboptimal transfer of nucleic acids from the RT-PCR step to the sequencing step, any non-specific RNA digestion during the RT-PCR step, or the presence of contaminating RNase during the RT reaction. A RT-PCR RNA stability ratio may be determined based on how much RNA is transferred. For example, the third plurality of nucleic acids (e.g., the RNA spike) may be added during the RT-PCR step in a known amount in a first location and then transferred to a second location for the sequencing step to be performed. If the transfer was effective, the RT-PCR RNA stability ratio will be higher, while the RT-PCR RNA stability ratio will be lower if the transfer is ineffective. For example, a RT-PCR RNA stability ratio of 0.3 will indicate that the transfer from the RT-PCR step to the sequencing step is ineffective, as only 30% of the RNA spikes made the transfer. By contrast, a RT-PCR RNA stability ratio of 0.99 will indicate that the transfer from the RT-PCR step to the sequencing step is effect, as 99% of the RNA spikes made the transfer. In the case of a low RT-PCR RNA stability ratio, certain conditions associated with the step may be changed, such as the speed of the transfer or the amount of RNA spike.

A fourth plurality of nucleic acids (e.g., DNA spike 610) can be added during the RT-PCR step. In certain embodiments, these nucleic acids comprise single-stranded DNA (ssDNA). This spike-in can be used to determine the effectiveness of the reverse transcriptase portion of the RT-PCR step. The fourth plurality of nucleic acids may include single stranded DNA spike added at the reverse transcriptase step, and read counts of the fourth plurality of nucleic acids at various steps may give insight as to how much of complementary DNA is being generated, thus indicating how effective the RT-PCR performance is and an RT efficiency ratio (e.g., “RT efficiency” 612). For example, a known amount of the fourth plurality of nucleic acids (e.g., the ssDNA spike) may be added during the RT-PCR step, and the known amount may be compared to a read count of the fourth plurality of nucleic acids during or after the RT-PCR step. For example, a RT efficiency ratio of 0.8 will indicate that the RT-PCR step converts 80% of the ssDNA. By contrast, a RT efficiency ratio of 0.1 will indicate that the RT-PCR step converts only 10% of the ssDNA. A low RT efficiency ratio may lead to changing of conditions of the RT-PCR step, such as temperature of the RT-PCR step, pH of the RT-PCR step, and/or duration of the RT-PCR step.

A fifth plurality of nucleic acids (e.g., RNA spike 606) can be added during a DNase step. In certain embodiments, these nucleic acids comprise RNA spike added at the DNase step. This spike-in can be used to determine the effectiveness of the liquid transfer from the DNase plates to the RT-PCR plates. A DNase RNA stability (e.g., “DNase RNA stability” 614) may be determined based on how much RNA is transferred. For example, the fifth plurality of nucleic acids (e.g., the RNA spike) may be added during the DNase step in a known amount in a first location (e.g., the DNase plates) and then transferred to a second location (e.g., the RT-PCR plates) for the reverse transcriptase step to be performed. If the transfer was effective, the DNase RNA stability ratio will be higher, while the DNase RNA stability ratio will be lower if the transfer is ineffective. For example, a DNase RNA stability ratio of 0.3 will indicate that the transfer from the DNase step to the reverse transcription step is ineffective, as only 30% of the RNA spikes made the transfer. By contrast, a DNase RNA stability ratio of 0.99 will indicate that the transfer from the DNase step to the reverse transcription step is effect, as 99% of the RNA spikes made the transfer. In the case of a low DNase RNA stability ratio, certain conditions associated with the step may be changed, such as the speed of the transfer or the amount of RNA spike.

Additional information may indicate further aspects associated with the process, such as additional ratios determined based on the pluralities of nucleic acids. For example, a gDNA contamination ratio (e.g., “gDNA contamination” 620) may be determined to indicate the gDNA contamination that arises after adding the first plurality of nucleic acids. For example, the first plurality of nucleic acids (e.g., double-stranded DNA spike) may be added to during the lysis step in a known amount, which may be compared to a read count of the first plurality of nucleic acids during the RT-PCR step to determine what percentage of DNA is gDNA. Thus, a gDNA contamination ratio of 0.75 will indicate that the DNase step converted a majority of the DNA spike ins that were added during the Lysis step into RNA. By contrast, a gDNA contamination ratio of 0.25 will indicate that the DNase step did convert a majority of the DNA spike ins that were added during the Lysis step into RNA.

A nucleotide sequence (e.g., barcode) of the control nucleic acids described herein can be changed for subsequent experimental runs to detect cross-over contamination from a previous experimental run. Such cross-over contamination can come from equipment or reagents used for a previous experimental run. In some embodiments, more than one plurality of nucleic acids may have the nucleotide sequenced in common. In other embodiments, each of the plurality of nucleic acids has its own nucleotide sequence.

Further, as described above, various pluralities of nucleic acids may be added during one or more steps. In some embodiments, a single plurality of nucleic acids may be added and evaluated through each step. For example, the single plurality of nucleic acids (e.g., RNA spike) may be added during the lysis step, counted at the end of the lysis step, counted at the beginning of the DNase step, counted at the end of the DNase step, counted at the beginning of the RT-PCR step, counted at the end of the RT-PCR step, and then counted once again during the sequencing step, effectively allowing for multiple read counts of the single plurality of nucleic acids to determine some of the above specified ratios, such as the Lysis RNA stability ratio, the DNase RNA stability ratio, and the RT-PCR RNA stability ratio.

In some embodiments, a plurality of nucleic acids may be present in solution at a concentration. In some embodiments, solvent may be added to achieve the concentration in solution. In some embodiments, the concentration may be about 0 pM to about 0.05 pM. In some embodiments, the concentration may be about 0 pM to about 0.01 pM, about 0 pM to about 0.015 pM, about 0 pM to about 0.02 pM, about 0 pM to about 0.025 pM, about 0 pM to about 0.03 pM, about 0 pM to about 0.035 pM, about 0 pM to about 0.04 pM, about 0 pM to about 0.045 pM, about 0 pM to about 0.05 pM, about 0.01 pM to about 0.015 pM, about 0.01 pM to about 0.02 pM, about 0.01 pM to about 0.025 pM, about 0.01 pM to about 0.03 pM, about 0.01 pM to about 0.035 pM, about 0.01 pM to about 0.04 pM, about 0.01 pM to about 0.045 pM, about 0.01 pM to about 0.05 pM, about 0.015 pM to about 0.02 pM, about 0.015 pM to about 0.025 pM, about 0.015 pM to about 0.03 pM, about 0.015 pM to about 0.035 pM, about 0.015 pM to about 0.04 pM, about 0.015 pM to about 0.045 pM, about 0.015 pM to about 0.05 pM, about 0.02 pM to about 0.025 pM, about 0.02 pM to about 0.03 pM, about 0.02 pM to about 0.035 pM, about 0.02 pM to about 0.04 pM, about 0.02 pM to about 0.045 pM, about 0.02 pM to about 0.05 pM, about 0.025 pM to about 0.03 pM, about 0.025 pM to about 0.035 pM, about 0.025 pM to about 0.04 pM, about 0.025 pM to about 0.045 pM, about 0.025 pM to about 0.05 pM, about 0.03 pM to about 0.035 pM, about 0.03 pM to about 0.04 pM, about 0.03 pM to about 0.045 pM, about 0.03 pM to about 0.05 pM, about 0.035 pM to about 0.04 pM, about 0.035 pM to about 0.045 pM, about 0.035 pM to about 0.05 pM, about 0.04 pM to about 0.045 pM, about 0.04 pM to about 0.05 pM, or about 0.045 pM to about 0.05 pM. In some embodiments, the concentration may be about 0 pM, about 0.01 pM, about 0.015 pM, about 0.02 pM, about 0.025 pM, about 0.03 pM, about 0.035 pM, about 0.04 pM, about 0.045 pM, or about 0.05 pM. In some embodiments, the concentration may be at least about 0 pM, about 0.01 pM, about 0.015 pM, about 0.02 pM, about 0.025 pM, about 0.03 pM, about 0.035 pM, about 0.04 pM, or about 0.045 pM. In some embodiments, the concentration may be at most about 0.01 pM, about 0.015 pM, about 0.02 pM, about 0.025 pM, about 0.03 pM, about 0.035 pM, about 0.04 pM, about 0.045 pM, or about 0.05 pM. In some embodiments, the concentration may be about 0.05 pM to about 0.5 pM. In some embodiments, the concentration may be about 0.05 pM to about 0.1 pM, about 0.05 pM to about 0.15 pM, about 0.05 pM to about 0.2 pM, about 0.05 pM to about 0.25 pM, about 0.05 pM to about 0.3 pM, about 0.05 pM to about 0.35 pM, about 0.05 pM to about 0.4 pM, about 0.05 pM to about 0.45 pM, about 0.05 pM to about 0.5 pM, about 0.1 pM to about 0.15 pM, about 0.1 pM to about 0.2 pM, about 0.1 pM to about 0.25 pM, about 0.1 pM to about 0.3 pM, about 0.1 pM to about 0.35 pM, about 0.1 pM to about 0.4 pM, about 0.1 pM to about 0.45 pM, about 0.1 pM to about 0.5 pM, about 0.15 pM to about 0.2 pM, about 0.15 pM to about 0.25 pM, about 0.15 pM to about 0.3 pM, about 0.15 pM to about 0.35 pM, about 0.15 pM to about 0.4 pM, about 0.15 pM to about 0.45 pM, about 0.15 pM to about 0.5 pM, about 0.2 pM to about 0.25 pM, about 0.2 pM to about 0.3 pM, about 0.2 pM to about 0.35 pM, about 0.2 pM to about 0.4 pM, about 0.2 pM to about 0.45 pM, about 0.2 pM to about 0.5 pM, about 0.25 pM to about 0.3 pM, about 0.25 pM to about 0.35 pM, about 0.25 pM to about 0.4 pM, about 0.25 pM to about 0.45 pM, about 0.25 pM to about 0.5 pM, about 0.3 pM to about 0.35 pM, about 0.3 pM to about 0.4 pM, about 0.3 pM to about 0.45 pM, about 0.3 pM to about 0.5 pM, about 0.35 pM to about 0.4 pM, about 0.35 pM to about 0.45 pM, about 0.35 pM to about 0.5 pM, about 0.4 pM to about 0.45 pM, about 0.4 pM to about 0.5 pM, or about 0.45 pM to about 0.5 pM. In some embodiments, the concentration may be about 0.05 pM, about 0.1 pM, about 0.15 pM, about 0.2 pM, about 0.25 pM, about 0.3 pM, about 0.35 pM, about 0.4 pM, about 0.45 pM, or about 0.5 pM. In some embodiments, the concentration may be at least about 0.05 pM, about 0.1 pM, about 0.15 pM, about 0.2 pM, about 0.25 pM, about 0.3 pM, about 0.35 pM, about 0.4 pM, or about 0.45 pM. In some embodiments, the concentration may be at most about 0.1 pM, about 0.15 pM, about 0.2 pM, about 0.25 pM, about 0.3 pM, about 0.35 pM, about 0.4 pM, about 0.45 pM, or about 0.5 pM. In some embodiments, the concentration may be about 0.5 pM to about 2 pM. In some embodiments, the concentration may be about 0.5 pM to about 0.6 pM, about 0.5 pM to about 0.7 pM, about 0.5 pM to about 0.8 pM, about 0.5 pM to about 0.9 pM, about 0.5 pM to about 1 pM, about 0.5 pM to about 1.25 pM, about 0.5 pM to about 1.5 pM, about 0.5 pM to about 1.75 pM, about 0.5 pM to about 2 pM, about 0.6 pM to about 0.7 pM, about 0.6 pM to about 0.8 pM, about 0.6 pM to about 0.9 pM, about 0.6 pM to about 1 pM, about 0.6 pM to about 1.25 pM, about 0.6 pM to about 1.5 pM, about 0.6 pM to about 1.75 pM, about 0.6 pM to about 2 pM, about 0.7 pM to about 0.8 pM, about 0.7 pM to about 0.9 pM, about 0.7 pM to about 1 pM, about 0.7 pM to about 1.25 pM, about 0.7 pM to about 1.5 pM, about 0.7 pM to about 1.75 pM, about 0.7 pM to about 2 pM, about 0.8 pM to about 0.9 pM, about 0.8 pM to about 1 pM, about 0.8 pM to about 1.25 pM, about 0.8 pM to about 1.5 pM, about 0.8 pM to about 1.75 pM, about 0.8 pM to about 2 pM, about 0.9 pM to about 1 pM, about 0.9 pM to about 1.25 pM, about 0.9 pM to about 1.5 pM, about 0.9 pM to about 1.75 pM, about 0.9 pM to about 2 pM, about 1 pM to about 1.25 pM, about 1 pM to about 1.5 pM, about 1 pM to about 1.75 pM, about 1 pM to about 2 pM, about 1.25 pM to about 1.5 pM, about 1.25 pM to about 1.75 pM, about 1.25 pM to about 2 pM, about 1.5 pM to about 1.75 pM, about 1.5 pM to about 2 pM, or about 1.75 pM to about 2 pM. In some embodiments, the concentration may be about 0.5 pM, about 0.6 pM, about 0.7 pM, about 0.8 pM, about 0.9 pM, about 1 pM, about 1.25 pM, about 1.5 pM, about 1.75 pM, or about 2 pM. In some embodiments, the concentration may be at least about 0.5 pM, about 0.6 pM, about 0.7 pM, about 0.8 pM, about 0.9 pM, about 1 pM, about 1.25 pM, about 1.5 pM, or about 1.75 pM. In some embodiments, the concentration may be at most about 0.6 pM, about 0.7 pM, about 0.8 pM, about 0.9 pM, about 1 pM, about 1.25 pM, about 1.5 pM, about 1.75 pM, or about 2 pM. In some embodiments, the concentration may be about 1 pM to about 50 pM. In some embodiments, the concentration may be about 1 pM to about 2 pM, about 1 pM to about 3 pM, about 1 pM to about 4 pM, about 1 pM to about 5 pM, about 1 pM to about 7.5 pM, about 1 pM to about 10 pM, about 1 pM to about 12.5 pM, about 1 pM to about 15 pM, about 1 pM to about 20 pM, about 1 pM to about 25 pM, about 1 pM to about 50 pM, about 2 pM to about 3 pM, about 2 pM to about 4 pM, about 2 pM to about 5 pM, about 2 pM to about 7.5 pM, about 2 pM to about 10 pM, about 2 pM to about 12.5 pM, about 2 pM to about 15 pM, about 2 pM to about 20 pM, about 2 pM to about 25 pM, about 2 pM to about 50 pM, about 3 pM to about 4 pM, about 3 pM to about 5 pM, about 3 pM to about 7.5 pM, about 3 pM to about 10 pM, about 3 pM to about 12.5 pM, about 3 pM to about 15 pM, about 3 pM to about 20 pM, about 3 pM to about 25 pM, about 3 pM to about 50 pM, about 4 pM to about 5 pM, about 4 pM to about 7.5 pM, about 4 pM to about 10 pM, about 4 pM to about 12.5 pM, about 4 pM to about 15 pM, about 4 pM to about 20 pM, about 4 pM to about 25 pM, about 4 pM to about 50 pM, about 5 pM to about 7.5 pM, about 5 pM to about 10 pM, about 5 pM to about 12.5 pM, about 5 pM to about 15 pM, about 5 pM to about 20 pM, about 5 pM to about 25 pM, about 5 pM to about 50 pM, about 7.5 pM to about 10 pM, about 7.5 pM to about 12.5 pM, about 7.5 pM to about 15 pM, about 7.5 pM to about 20 pM, about 7.5 pM to about 25 pM, about 7.5 pM to about 50 pM, about 10 pM to about 12.5 pM, about 10 pM to about 15 pM, about 10 pM to about 20 pM, about 10 pM to about 25 pM, about 10 pM to about 50 pM, about 12.5 pM to about 15 pM, about 12.5 pM to about 20 pM, about 12.5 pM to about 25 pM, about 12.5 pM to about 50 pM, about 15 pM to about 20 pM, about 15 pM to about 25 pM, about 15 pM to about 50 pM, about 20 pM to about 25 pM, about 20 pM to about 50 pM, or about 25 pM to about 50 pM. In some embodiments, the concentration may be about 1 pM, about 2 pM, about 3 pM, about 4 pM, about 5 pM, about 7.5 pM, about 10 pM, about 12.5 pM, about 15 pM, about 20 pM, about 25 pM, or about 50 pM. In some embodiments, the concentration may be at least about 1 pM, about 2 pM, about 3 pM, about 4 pM, about 5 pM, about 7.5 pM, about 10 pM, about 12.5 pM, about 15 pM, about 20 pM, or about 25 pM. In some embodiments, the concentration may be at most about 2 pM, about 3 pM, about 4 pM, about 5 pM, about 7.5 pM, about 10 pM, about 12.5 pM, about 15 pM, about 20 pM, about 25 pM, or about 50 pM. While certain concentrations are described above, the concentrations are exemplary, and other concentrations may be used. Various nucleic acids may be present at various concentrations in a well. Further, not all nucleic acids need to be present in the same concentrations between wells.

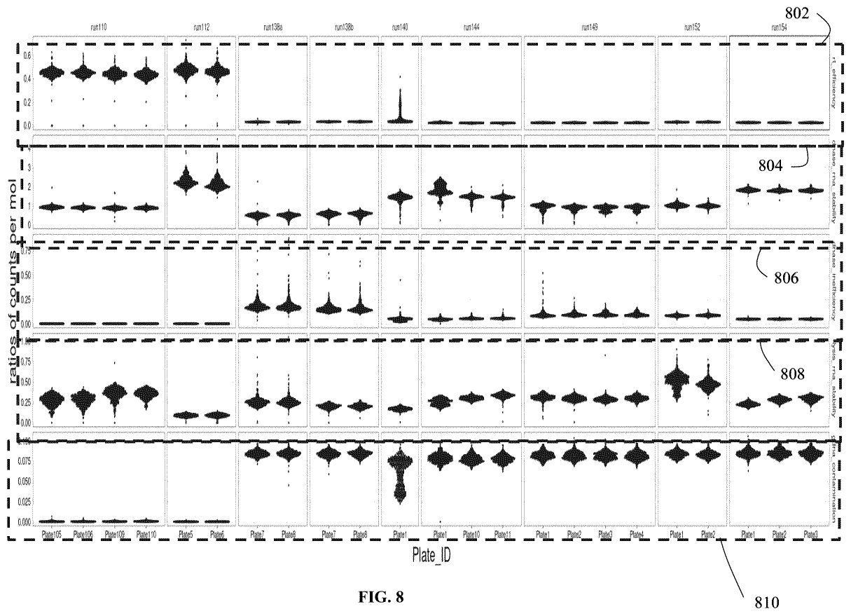

FIG. 8 shows the ratios of read counts per mol for various wells of multiple assays.

Section 802 shows RT efficiency ratios between 0.0 and 1.0 for the various wells, with an expected RT efficiency ratio of 0.50. In particular, if an RT efficiency ratio is lower than a 0.39, the RT efficiency ratio indicates that the data is unreliable, and can be excluded. RT efficiency ratios indicate the effectiveness of the reverse transcriptase portion of the RT-PCR step (e.g., what portion of a plurality of nucleic acids (e.g., ssDNA spike ins) are converted by the reverse transcriptase). A higher RT efficiency ratio indicates more reliable data, while a lower RT efficiency ratio indicates that the data may need to be excluded. In some embodiments, the RT efficiency ratio may be about 0 to about 1. In some embodiments, the RT efficiency ratio may be about 0 to about 0.1, about 0 to about 0.2, about 0 to about 0.3, about 0 to about 0.4, about 0 to about 0.5, about 0 to about 0.6, about 0 to about 0.7, about 0 to about 0.8, about 0 to about 0.9, about 0 to about 0.95, about 0 to about 1, about 0.1 to about 0.2, about 0.1 to about 0.3, about 0.1 to about 0.4, about 0.1 to about 0.5, about 0.1 to about 0.6, about 0.1 to about 0.7, about 0.1 to about 0.8, about 0.1 to about 0.9, about 0.1 to about 0.95, about 0.1 to about 1, about 0.2 to about 0.3, about 0.2 to about 0.4, about 0.2 to about 0.5, about 0.2 to about 0.6, about 0.2 to about 0.7, about 0.2 to about 0.8, about 0.2 to about 0.9, about 0.2 to about 0.95, about 0.2 to about 1, about 0.3 to about 0.4, about 0.3 to about 0.5, about 0.3 to about 0.6, about 0.3 to about 0.7, about 0.3 to about 0.8, about 0.3 to about 0.9, about 0.3 to about 0.95, about 0.3 to about 1, about 0.4 to about 0.5, about 0.4 to about 0.6, about 0.4 to about 0.7, about 0.4 to about 0.8, about 0.4 to about 0.9, about 0.4 to about 0.95, about 0.4 to about 1, about 0.5 to about 0.6, about 0.5 to about 0.7, about 0.5 to about 0.8, about 0.5 to about 0.9, about 0.5 to about 0.95, about 0.5 to about 1, about 0.6 to about 0.7, about 0.6 to about 0.8, about 0.6 to about 0.9, about 0.6 to about 0.95, about 0.6 to about 1, about 0.7 to about 0.8, about 0.7 to about 0.9, about 0.7 to about 0.95, about 0.7 to about 1, about 0.8 to about 0.9, about 0.8 to about 0.95, about 0.8 to about 1, about 0.9 to about 0.95, about 0.9 to about 1, or about 0.95 to about 1. In some embodiments, the RT efficiency ratio may be about 0, about 0.1, about 0.2, about 0.3, about 0.4, about 0.5, about 0.6, about 0.7, about 0.8, about 0.9, about 0.95, or about 1. In some embodiments, the RT efficiency ratio may be at least about 0, about 0.1, about 0.2, about 0.3, about 0.4, about 0.5, about 0.6, about 0.7, about 0.8, about 0.9, or about 0.95. In some embodiments, the RT efficiency ratio may be at most about 0.1, about 0.2, about 0.3, about 0.4, about 0.5, about 0.6, about 0.7, about 0.8, about 0.9, about 0.95, or about 1.

Section 804 shows DNase-RNA stability ratios of between 0.00 and 4.00 for the various wells, with an expected DNase-RNA stability ratio of between 1.00 to 2.00. In particular, if a DNase stability ratio is higher than 2.75 or lower than 1.75, the DNase stability ratio indicates that the data may be unreliable, and can be excluded. DNase-RNA stability ratios indicate how many of a plurality of nucleic acids that are added during the DNase step remain after a transfer to another well (e.g., by comparing the amount of nucleic acids added during the step to the amount of nucleic acids remaining after the DNase step). A higher than expected DNase-RNA stability ratio indicates there may have been corruption of data (e.g., data indicating that later steps have been affected by the addition of nucleic acids), while a lower than expected DNase-RNA stability ratio indicates that the transfer was unsuccessful. In some embodiments, the DNase RNA stability ratio may be about 0 to about 2.5. In some embodiments, the DNase RNA stability ratio may be about 0 to about 0.25, about 0 to about 0.5, about 0 to about 0.75, about 0 to about 1, about 0 to about 1.25, about 0 to about 1.5, about 0 to about 1.75, about 0 to about 2, about 0 to about 2.25, about 0 to about 2.5, about 0.25 to about 0.5, about 0.25 to about 0.75, about 0.25 to about 1, about 0.25 to about 1.25, about 0.25 to about 1.5, about 0.25 to about 1.75, about 0.25 to about 2, about 0.25 to about 2.25, about 0.25 to about 2.5, about 0.5 to about 0.75, about 0.5 to about 1, about 0.5 to about 1.25, about 0.5 to about 1.5, about 0.5 to about 1.75, about 0.5 to about 2, about 0.5 to about 2.25, about 0.5 to about 2.5, about 0.75 to about 1, about 0.75 to about 1.25, about 0.75 to about 1.5, about 0.75 to about 1.75, about 0.75 to about 2, about 0.75 to about 2.25, about 0.75 to about 2.5, about 1 to about 1.25, about 1 to about 1.5, about 1 to about 1.75, about 1 to about 2, about 1 to about 2.25, about 1 to about 2.5, about 1.25 to about 1.5, about 1.25 to about 1.75, about 1.25 to about 2, about 1.25 to about 2.25, about 1.25 to about 2.5, about 1.5 to about 1.75, about 1.5 to about 2, about 1.5 to about 2.25, about 1.5 to about 2.5, about 1.75 to about 2, about 1.75 to about 2.25, about 1.75 to about 2.5, about 2 to about 2.25, about 2 to about 2.5, or about 2.25 to about 2.5. In some embodiments, the DNase RNA stability ratio may be about 0, about 0.25, about 0.5, about 0.75, about 1, about 1.25, about 1.5, about 1.75, about 2, about 2.25, or about 2.5. In some embodiments, the DNase RNA stability ratio may be at least about 0, about 0.25, about 0.5, about 0.75, about 1, about 1.25, about 1.5, about 1.75, about 2, or about 2.25. In some embodiments, the DNase RNA stability ratio may be at most about 0.25, about 0.5, about 0.75, about 1, about 1.25, about 1.5, about 1.75, about 2, about 2.25, or about 2.5. In some embodiments, the DNase RNA stability ratio may be about 1.75 to about 2.75. In some embodiments, the DNase RNA stability ratio may be about 1.75 to about 2, about 1.75 to about 2.25, about 1.75 to about 2.5, about 1.75 to about 2.75, about 2 to about 2.25, about 2 to about 2.5, about 2 to about 2.75, about 2.25 to about 2.5, about 2.25 to about 2.75, or about 2.5 to about 2.75. In some embodiments, the DNase RNA stability ratio may be about 1.75, about 2, about 2.25, about 2.5, or about 2.75. In some embodiments, the DNase RNA stability ratio may be at least about 1.75, about 2, about 2.25, or about 2.5. In some embodiments, the DNase RNA stability ratio may be at most about 2, about 2.25, about 2.5, or about 2.75. In some embodiments, the DNase RNA stability ratio may be about 2.75 to about 4. In some embodiments, the DNase RNA stability ratio may be about 2.75 to about 3, about 2.75 to about 3.25, about 2.75 to about 3.5, about 2.75 to about 3.75, about 2.75 to about 4, about 3 to about 3.25, about 3 to about 3.5, about 3 to about 3.75, about 3 to about 4, about 3.25 to about 3.5, about 3.25 to about 3.75, about 3.25 to about 4, about 3.5 to about 3.75, about 3.5 to about 4, or about 3.75 to about 4. In some embodiments, the DNase RNA stability ratio may be about 2.75, about 3, about 3.25, about 3.5, about 3.75, or about 4. In some embodiments, the DNase RNA stability ratio may be at least about 2.75, about 3, about 3.25, about 3.5, or about 3.75. In some embodiments, the DNase RNA stability ratio may be at most about 3, about 3.25, about 3.5, about 3.75, or about 4. In some embodiments, the DNase RNA stability ratio may be about 2 to about 20. In some embodiments, the DNase RNA stability ratio may be about 2 to about 4, about 2 to about 6, about 2 to about 8, about 2 to about 10, about 2 to about 12, about 2 to about 14, about 2 to about 16, about 2 to about 18, about 2 to about 20, about 4 to about 6, about 4 to about 8, about 4 to about 10, about 4 to about 12, about 4 to about 14, about 4 to about 16, about 4 to about 18, about 4 to about 20, about 6 to about 8, about 6 to about 10, about 6 to about 12, about 6 to about 14, about 6 to about 16, about 6 to about 18, about 6 to about 20, about 8 to about 10, about 8 to about 12, about 8 to about 14, about 8 to about 16, about 8 to about 18, about 8 to about 20, about 10 to about 12, about 10 to about 14, about 10 to about 16, about 10 to about 18, about 10 to about 20, about 12 to about 14, about 12 to about 16, about 12 to about 18, about 12 to about 20, about 14 to about 16, about 14 to about 18, about 14 to about 20, about 16 to about 18, about 16 to about 20, or about 18 to about 20. In some embodiments, the DNase RNA stability ratio may be about 2, about 4, about 6, about 8, about 10, about 12, about 14, about 16, about 18, or about 20. In some embodiments, the DNase RNA stability ratio may be at least about 2, about 4, about 6, about 8, about 10, about 12, about 14, about 16, or about 18. In some embodiments, the DNase RNA stability ratio may be at most about 4, about 6, about 8, about 10, about 12, about 14, about 16, about 18, or about 20.

Section 806 shows DNase inefficiency ratios of between 0.00 and 0.75 for the various wells, with an expected DNase inefficiency ratio of around 0.05. In particular, if a DNase inefficiency ratio is higher than 0.15, the DNase inefficiency ratio indicates that the data is unreliable, and can be excluded. A DNase inefficiency indicates an effectiveness of the DNase step by comparing a remaining amount of nucleic acids added during the lysis step (e.g., DNA spike ins) to an amount of nucleic acids added during the DNase step (e.g., RNA spike ins). A higher DNase inefficiency ratio indicates that the DNase step was not successful, and the associated data should be excluded. In some embodiments, the DNase inefficiency ratio may be about 0 to about 0.1. In some embodiments, the DNase inefficiency ratio may be about 0 to about 0.01, about 0 to about 0.02, about 0 to about 0.03, about 0 to about 0.04, about 0 to about 0.05, about 0 to about 0.06, about 0 to about 0.07, about 0 to about 0.08, about 0 to about 0.09, about 0 to about 0.1, about 0.01 to about 0.02, about 0.01 to about 0.03, about 0.01 to about 0.04, about 0.01 to about 0.05, about 0.01 to about 0.06, about 0.01 to about 0.07, about 0.01 to about 0.08, about 0.01 to about 0.09, about 0.01 to about 0.1, about 0.02 to about 0.03, about 0.02 to about 0.04, about 0.02 to about 0.05, about 0.02 to about 0.06, about 0.02 to about 0.07, about 0.02 to about 0.08, about 0.02 to about 0.09, about 0.02 to about 0.1, about 0.03 to about 0.04, about 0.03 to about 0.05, about 0.03 to about 0.06, about 0.03 to about 0.07, about 0.03 to about 0.08, about 0.03 to about 0.09, about 0.03 to about 0.1, about 0.04 to about 0.05, about 0.04 to about 0.06, about 0.04 to about 0.07, about 0.04 to about 0.08, about 0.04 to about 0.09, about 0.04 to about 0.1, about 0.05 to about 0.06, about 0.05 to about 0.07, about 0.05 to about 0.08, about 0.05 to about 0.09, about 0.05 to about 0.1, about 0.06 to about 0.07, about 0.06 to about 0.08, about 0.06 to about 0.09, about 0.06 to about 0.1, about 0.07 to about 0.08, about 0.07 to about 0.09, about 0.07 to about 0.1, about 0.08 to about 0.09, about 0.08 to about 0.1, or about 0.09 to about 0.1. In some embodiments, the DNase inefficiency ratio may be about 0, about 0.01, about 0.02, about 0.03, about 0.04, about 0.05, about 0.06, about 0.07, about 0.08, about 0.09, or about 0.1. In some embodiments, the DNase inefficiency ratio may be at least about 0, about 0.01, about 0.02, about 0.03, about 0.04, about 0.05, about 0.06, about 0.07, about 0.08, or about 0.09. In some embodiments, the DNase inefficiency ratio may be at most about 0.01, about 0.02, about 0.03, about 0.04, about 0.05, about 0.06, about 0.07, about 0.08, about 0.09, or about 0.1. In some embodiments, the DNase inefficiency ratio may be about 0 to about 1. In some embodiments, the DNase inefficiency ratio may be about 0 to about 0.1, about 0 to about 0.2, about 0 to about 0.3, about 0 to about 0.4, about 0 to about 0.5, about 0 to about 0.6, about 0 to about 0.7, about 0 to about 0.8, about 0 to about 0.9, about 0 to about 1, about 0.1 to about 0.2, about 0.1 to about 0.3, about 0.1 to about 0.4, about 0.1 to about 0.5, about 0.1 to about 0.6, about 0.1 to about 0.7, about 0.1 to about 0.8, about 0.1 to about 0.9, about 0.1 to about 1, about 0.2 to about 0.3, about 0.2 to about 0.4, about 0.2 to about 0.5, about 0.2 to about 0.6, about 0.2 to about 0.7, about 0.2 to about 0.8, about 0.2 to about 0.9, about 0.2 to about 1, about 0.3 to about 0.4, about 0.3 to about 0.5, about 0.3 to about 0.6, about 0.3 to about 0.7, about 0.3 to about 0.8, about 0.3 to about 0.9, about 0.3 to about 1, about 0.4 to about 0.5, about 0.4 to about 0.6, about 0.4 to about 0.7, about 0.4 to about 0.8, about 0.4 to about 0.9, about 0.4 to about 1, about 0.5 to about 0.6, about 0.5 to about 0.7, about 0.5 to about 0.8, about 0.5 to about 0.9, about 0.5 to about 1, about 0.6 to about 0.7, about 0.6 to about 0.8, about 0.6 to about 0.9, about 0.6 to about 1, about 0.7 to about 0.8, about 0.7 to about 0.9, about 0.7 to about 1, about 0.8 to about 0.9, about 0.8 to about 1, or about 0.9 to about 1. In some embodiments, the DNase inefficiency ratio may be about 0, about 0.1, about 0.2, about 0.3, about 0.4, about 0.5, about 0.6, about 0.7, about 0.8, about 0.9, or about 1. In some embodiments, the DNase inefficiency ratio may be at least about 0, about 0.1, about 0.2, about 0.3, about 0.4, about 0.5, about 0.6, about 0.7, about 0.8, or about 0.9. In some embodiments, the DNase inefficiency ratio may be at most about 0.1, about 0.2, about 0.3, about 0.4, about 0.5, about 0.6, about 0.7, about 0.8, about 0.9, or about 1.

Section 808 shows Lysis RNA stability ratio of between 0.00 and 1.00 for the various wells, with an expected Lysis RNA stability ratio of between 0.20 and 0.50. In particular, if a Lysis RNA stability ratio is lower than 0.15, the Lysis RNA stability ratio indicates that the data is unreliable, and can be excluded. A Lysis RNA stability ratio indicates the effectiveness of a transfer between steps or determine if the presence of RNase impacts later steps by comparing an amount of nucleic acids added during the lysis step (e.g., RNA spike ins) to an amount of nucleic acids added during the DNase step (e.g., other RNA spike ins). A higher than expected Lysis RNA stability ratio indicates there may have been corruption of data (e.g., data indicating that later steps have been affected by the addition of nucleic acids), while a lower than expected Lysis RNA stability ratio indicates that the transfer was unsuccessful. In some embodiments, the Lysis RNA stability ratio may be about 0 to about 1. In some embodiments, the Lysis RNA stability ratio may be about 0 to about 0.1, about 0 to about 0.2, about 0 to about 0.3, about 0 to about 0.4, about 0 to about 0.5, about 0 to about 0.6, about 0 to about 0.7, about 0 to about 0.8, about 0 to about 0.9, about 0 to about 1, about 0.1 to about 0.2, about 0.1 to about 0.3, about 0.1 to about 0.4, about 0.1 to about 0.5, about 0.1 to about 0.6, about 0.1 to about 0.7, about 0.1 to about 0.8, about 0.1 to about 0.9, about 0.1 to about 1, about 0.2 to about 0.3, about 0.2 to about 0.4, about 0.2 to about 0.5, about 0.2 to about 0.6, about 0.2 to about 0.7, about 0.2 to about 0.8, about 0.2 to about 0.9, about 0.2 to about 1, about 0.3 to about 0.4, about 0.3 to about 0.5, about 0.3 to about 0.6, about 0.3 to about 0.7, about 0.3 to about 0.8, about 0.3 to about 0.9, about 0.3 to about 1, about 0.4 to about 0.5, about 0.4 to about 0.6, about 0.4 to about 0.7, about 0.4 to about 0.8, about 0.4 to about 0.9, about 0.4 to about 1, about 0.5 to about 0.6, about 0.5 to about 0.7, about 0.5 to about 0.8, about 0.5 to about 0.9, about 0.5 to about 1, about 0.6 to about 0.7, about 0.6 to about 0.8, about 0.6 to about 0.9, about 0.6 to about 1, about 0.7 to about 0.8, about 0.7 to about 0.9, about 0.7 to about 1, about 0.8 to about 0.9, about 0.8 to about 1, or about 0.9 to about 1. In some embodiments, the Lysis RNA stability ratio may be about 0, about 0.1, about 0.2, about 0.3, about 0.4, about 0.5, about 0.6, about 0.7, about 0.8, about 0.9, or about 1. In some embodiments, the Lysis RNA stability ratio may be at least about 0, about 0.1, about 0.2, about 0.3, about 0.4, about 0.5, about 0.6, about 0.7, about 0.8, or about 0.9. In some embodiments, the Lysis RNA stability ratio may be at most about 0.1, about 0.2, about 0.3, about 0.4, about 0.5, about 0.6, about 0.7, about 0.8, about 0.9, or about 1.

Section 810 shows gDNA contamination ratio of between 0.00 and 0.10 for the various wells, with an expected gDNA contamination ratio of between 0.0 and 0.09. In particular, if a gDNA contamination ratio is higher than 0.10, the gDNA contamination ratio indicates that the data is unreliable, and can be excluded. The gDNA contamination ratio indicates how much DNA was not converted over the course of the assay by comparing a plurality of nucleic acids added during the lysis step (e.g., DNA spike ins) to a plurality of nucleic acids added during the RT-PCR step before sequencing (e.g., another plurality of DNA spike ins). A higher gDNA contamination ratio indicates that the addition of previous nucleic acids affected later steps, and thus, the data may need to be excluded. In some embodiments, the gDNA contamination ratio may be about 0 to about 0.5. In some embodiments, the gDNA contamination ratio may be about 0 to about 0.05, about 0 to about 0.1, about 0 to about 0.15, about 0 to about 0.2, about 0 to about 0.25, about 0 to about 0.3, about 0 to about 0.35, about 0 to about 0.4, about 0 to about 0.45, about 0 to about 0.5, about 0.05 to about 0.1, about 0.05 to about 0.15, about 0.05 to about 0.2, about 0.05 to about 0.25, about 0.05 to about 0.3, about 0.05 to about 0.35, about 0.05 to about 0.4, about 0.05 to about 0.45, about 0.05 to about 0.5, about 0.1 to about 0.15, about 0.1 to about 0.2, about 0.1 to about 0.25, about 0.1 to about 0.3, about 0.1 to about 0.35, about 0.1 to about 0.4, about 0.1 to about 0.45, about 0.1 to about 0.5, about 0.15 to about 0.2, about 0.15 to about 0.25, about 0.15 to about 0.3, about 0.15 to about 0.35, about 0.15 to about 0.4, about 0.15 to about 0.45, about 0.15 to about 0.5, about 0.2 to about 0.25, about 0.2 to about 0.3, about 0.2 to about 0.35, about 0.2 to about 0.4, about 0.2 to about 0.45, about 0.2 to about 0.5, about 0.25 to about 0.3, about 0.25 to about 0.35, about 0.25 to about 0.4, about 0.25 to about 0.45, about 0.25 to about 0.5, about 0.3 to about 0.35, about 0.3 to about 0.4, about 0.3 to about 0.45, about 0.3 to about 0.5, about 0.35 to about 0.4, about 0.35 to about 0.45, about 0.35 to about 0.5, about 0.4 to about 0.45, about 0.4 to about 0.5, or about 0.45 to about 0.5. In some embodiments, the gDNA contamination ratio may be about 0, about 0.05, about 0.1, about 0.15, about 0.2, about 0.25, about 0.3, about 0.35, about 0.4, about 0.45, or about 0.5. In some embodiments, the gDNA contamination ratio may be at least about 0, about 0.05, about 0.1, about 0.15, about 0.2, about 0.25, about 0.3, about 0.35, about 0.4, or about 0.45. In some embodiments, the gDNA contamination ratio may be at most about 0.05, about 0.1, about 0.15, about 0.2, about 0.25, about 0.3, about 0.35, about 0.4, about 0.45, or about 0.5.

While certain ratios are shown and certain expected ratios are described, a skilled artisan would be able to determine ratios from read counts of nucleic acids, as well as expected ratios based on expected read counts. Additionally, while certain parameters for unreliable data are described, these are exemplary, and may change based on what steps are performed and in what conditions the steps are performed and the ultimate experimental goals of the artisan.

### Barcodes

Variable nucleotide sequences (barcodes) that serve as an index can be included on any of the reporter genes described herein. Additionally, barcodes may be added in a separate library preparation reaction. The variable nucleotide sequences described herein can be used as a sample index in order to deconvolve results obtained from a sequencing reaction used herein.

Once the contents of the cells are released into their respective partitions by a lysis agent, the macromolecular components (e.g., macromolecular constituents of samples, such as RNA, DNA, or proteins) contained therein may be further processed within the partitions. In accordance with the methods and systems described herein, the macromolecular component contents of individual samples can be provided with unique identifiers such that, upon characterization of those macromolecular components they may be attributed as having been derived from the same sample or particles. The ability to attribute characteristics to individual samples or groups of samples is provided by the assignment of unique identifiers specifically to an individual sample or groups of samples. Unique identifiers, e.g., in the form of nucleic acid barcodes can be assigned or associated with individual samples or populations of samples, in order to tag or label the sample's macromolecular components (and as a result, its characteristics) with the unique identifiers. These unique identifiers can then be used to attribute the sample's components and characteristics to an individual sample or group of samples.

In some aspects, this is performed by co-partitioning the individual sample or groups of samples with the unique identifiers or barcodes comprising an unique molecular identifier sequence (UMI). In some aspects, the unique identifiers are provided in the form of nucleic acid molecules (e.g., oligonucleotides) that comprise nucleic acid barcode sequences that may be attached to or otherwise associated with the nucleic acid contents of individual sample, or to other components of the sample, and particularly to fragments of those nucleic acids. The nucleic acid molecules are partitioned such that as between nucleic acid molecules in a given partition, the nucleic acid barcode sequences contained therein are the same, but as between different partitions, the nucleic acid molecule can, and do have differing barcode sequences, or at least represent a large number of different barcode sequences across all of the partitions in a given analysis. In some aspects, only one nucleic acid barcode sequence can be associated with a given partition, although in some embodiments, two or more different barcode sequences may be present.

The nucleic acid barcode sequences can include from about 6 to about 20 or more nucleotides within the sequence of the nucleic acid molecules (e.g., oligonucleotides). The nucleic acid barcode sequences can include from about 6 to about 20, 30, 40, 50, 60, 70, 80, 90, 100 or more nucleotides. In some embodiments, the length of a barcode sequence may be about 6, 7, 8, 9, 10, 11, 12, 13, 14, 15, 16, 17, 18, 19, 20 nucleotides or longer. In some embodiments, the length of a barcode sequence may be at least about 6, 7, 8, 9, 10, 11, 12, 13, 14, 15, 16, 17, 18, 19, 20 nucleotides or longer. In some embodiments, the length of a barcode sequence may be at most about 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In some embodiments, separated barcode subsequences can be from about 4 to about 16 nucleotides in length. In some embodiments, the barcode subsequence may be about 4, 5, 6, 7, 8, 9, 10, 11, 12, 13, 14, 15, 16 nucleotides or longer. In some embodiments, the barcode subsequence may be at least about 4, 5, 6, 7, 8, 9, 10, 11, 12, 13, 14, 15, 16 nucleotides or longer. In some embodiments, the barcode subsequence may be at most about 4, 5, 6, 7, 8, 9, 10, 11, 12, 13, 14, 15, 16 nucleotides or shorter.

The co-partitioned nucleic acid molecules can also comprise other functional sequences useful in the processing of the nucleic acids from the co-partitioned samples. These sequences include, e.g., targeted or random/universal amplification primer sequences for amplifying the genomic DNA from the individual samples within the partitions while attaching the associated barcode sequences, sequencing primers or primer recognition sites, hybridization or probing sequences, e.g., for identification of presence of the sequences or for pulling down barcoded nucleic acids, or any of a number of other potential functional sequences. Other mechanisms of co-partitioning oligonucleotides may also be employed, including, e.g., coalescence of two or more partitions, where one partition contains oligonucleotides, or microdispensing of oligonucleotides into partitions, e.g., partitions within microfluidic systems. In some embodiments, a primer comprises a barcode oligonucleotide. In some embodiments the primer sequence is a targeted primer sequence complementary to a sequence in the template nucleic acid molecule. In some embodiments, the first nucleic acid molecule further comprises one or more functional sequences and wherein the second nucleic acid molecule comprises the one or more functional sequences. In some embodiments, the one or more functional sequences are selected from the group consisting of an adapter sequence, an additional primer sequence, a primer annealing sequence, a sequencing primer sequence, a sequence configured to attach to a flow cell of a sequencer, and a unique molecular identifier sequence.

For example, the above described barcoded nucleic acid molecules (e.g., barcoded oligonucleotides) are added to a sample. In some embodiments, a partition comprises barcoded oligonucleotides having the same barcode sequence. In some embodiments, a partition among a plurality of partitions comprises barcoded oligonucleotides having an identical barcode sequence, wherein each partition among within the plurality of partitions comprises a unique barcode sequence. In some embodiments, the population of barcoded oligonucleotides provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more. Additionally, each barcoded oligonucleotide can be provided with large numbers of nucleic acid (e.g., oligonucleotide) molecules attached. In particular, the number of molecules of nucleic acid molecules including the barcode sequence on an individual barcoded oligonucleotide can be at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some embodiments at least about 1 billion nucleic acid molecules, or more. Nucleic acid molecules of a given barcoded oligonucleotide can include identical (or common) barcode sequences, different barcode sequences, or a combination of both. Nucleic acid molecules of a given barcoded oligonucleotide can include multiple sets of nucleic acid molecules. Nucleic acid molecules of a given set can include identical barcode sequences. The identical barcode sequences can be different from barcode sequences of nucleic acid molecules of another set

Moreover, when the population of barcoded oligonucleotides is partitioned, the resulting population of partitions can also include a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each partition of the population can include at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some embodiments at least about 1 billion nucleic acid molecules.

In some embodiments, it may be desirable to incorporate multiple different barcodes within a given partition. For example, in some embodiments, a barcoded oligonucleotide within a partition can comprise (1) a common barcode sequence shared by all barcoded oligonucleotides within the partition and (2) a unique molecular identifier or additional barcode sequence that is different among each barcoded oligonucleotide. The common barcode sequences may provide greater assurance of identification in the subsequent processing, e.g., by providing a stronger address or attribution of the barcodes to a given partition, as a duplicate or independent confirmation of the output from a given partition.

In some embodiments, the barcoded oligonucleotides are attached to the beads, where all of the nucleic acid molecules attached to a particular bead will include the same nucleic acid barcode sequence, but where a large number of diverse barcode sequences are represented across the population of beads used. In some embodiments, hydrogel beads, e.g., comprising polyacrylamide polymer matrices, are used as a solid support and delivery vehicle for the nucleic acid molecules into the partitions, as they are capable of carrying large numbers of nucleic acid molecules, and may be configured to release those nucleic acid molecules upon exposure to a particular stimulus, as described elsewhere herein.

The nucleic acid molecules (e.g., oligonucleotides) can be releasable from the beads upon the application of a particular stimulus to the beads. In some embodiments, the stimulus may be a photo-stimulus, e.g., through cleavage of a photo-labile linkage that releases the nucleic acid molecules. In other embodiments, a thermal stimulus may be used, where elevation of the temperature of the beads environment will result in cleavage of a linkage or other release of the nucleic acid molecules form the beads. In still other embodiments, a chemical stimulus can be used that cleaves a linkage of the nucleic acid molecules to the beads, or otherwise results in release of the nucleic acid molecules from the beads. In one embodiment, such compositions include the polyacrylamide matrices described above for encapsulation of samples, and may be degraded for release of the attached nucleic acid molecules through exposure to a reducing agent, such as DTT.

A support can be contemplated for use in a method of the present disclosure may be, for example, a well, matrix, rod, container, or bead(s). A support may have any useful features and characteristics, such as any useful size, surface chemistry, fluidity, solidity, density, porosity, and composition. In some embodiments, a support is a surface of a well on a plate. In some embodiments, a support may be a bead such as a gel bead. A bead may be solid or semi-solid. Additional details of beads are provided elsewhere herein.

A support (e.g., a bead) may comprise an anchor sequence functionalized thereto (e.g., as described herein). An anchor sequence may be attached to the support via, for example, a disulfide linkage. An anchor sequence may comprise a partial read sequence and/or flow cell functional sequence. Such a sequence may permit sequencing of nucleic acid molecules attached to the sequence by a sequencer (e.g., an Illumina sequencer). Different anchor sequences may be useful for different sequencing applications. An anchor sequence may comprise, for example, a TruSeq or Nextera sequence. An anchor sequence may have any useful characteristics such as any useful length and nucleotide composition. For example, an anchor sequence may comprise 2, 3, 4, 5, 6, 7, 8, 9, 10, 11, 12, 13, 14, 15, 16, 17, 18, 19, 20, or more nucleotides. In some embodiments, an anchor sequence may comprise 15 nucleotides. Nucleotides of an anchor sequence may be naturally occurring or non-naturally occurring (e.g., as described herein). A bead may comprise a plurality of anchor sequences attached thereto. For example, a bead may comprise a plurality of first anchor sequences attached thereto. In some embodiments, a bead may comprise two or more different anchor sequences attached thereto. For example, a bead may comprise both a plurality of first anchor sequences (e.g., Nextera sequences) and a plurality of second anchor sequences (e.g., TruSeq sequences) attached thereto. For a bead comprising two or more different anchor sequences attached thereto, the sequence of each different anchor sequence may be distinguishable from the sequence of each other anchor sequence at an end distal to the bead. For example, the different anchor sequences may comprise one or more nucleotide differences in the 2, 3, 4, 5, 6, 7, 8, 9, 10, or more nucleotides furthest from the bead.

In some embodiments, multiple different barcode molecules (e.g., nucleic acid barcode molecules) may be generated on the same support (e.g., bead). For example, two different barcode molecules may be generated on the same support. Alternatively, three or more different barcode molecules may be generated on the same support. Different barcode molecules attached to the same support may comprise one or more different sequences. For example, different barcode molecules may comprise one or more different barcode sequences, and/or other sequences (e.g., starter sequences). In some embodiments, different barcode molecules attached to the same support may comprise the same barcode sequences. Different barcode molecules attached to the same support may comprise barcode sequences that are the same or different. Similarly, different barcode molecules may comprise unique molecular identifiers (UMIs) that are the same or different.

### Next Generation Sequencing

As described in the methods disclosed herein, the sequencing of nucleic acid molecules is used and is useful for the detection of biological effect by a test agent against a cell comprising a cell based assay. Generally, sequencing refers to methods and technologies for determining the sequence of nucleotide bases in one or more polynucleotides. The polynucleotides can be, for example, deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina, Pacific Biosciences, Oxford Nanopore, or Life Technologies (Ion Torrent). Such devices may provide a plurality of raw genetic data corresponding to the genetic information of a subject (e.g., human), as generated by the device from a sample provided by the subject. In some situations, systems and methods provided herein may be used with proteomic information. Alternatively, or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

Next generation sequencing includes many technologies capable of generating large amounts of sequence information (e.g., high-throughput sequencing) and excluding Sanger sequencing or Maxam-Gilbert sequencing. Generally, next generation sequencing encompasses single molecule real-time sequencing, sequencing-by-synthesis, ion semiconductor sequencing and the like. Exemplary next-generation sequencing machines may comprise the MiniSeq, the iSeq100, the NextSeq 1000, the NextSeq 2000, the NovaSeq 6000, the NextSeq 550 series and the like from Illumina, Inc; Ion Torrent machines from Thermo Fisher Scientific; or the Sequel systems from Pacific Biosciences.

Next generation sequencing machines used with the method herein can generate at least 1, 5, 10, 15, 25, 50, 75, 100, 200, 300 gigabases of data or more in a 24 hour period from a single machine.

Next generation sequencing machines used with the method herein can generate at least 1, 1, 4, 10, 15, 25, 50, 75, 100, 200, 300, 500, or 1,000 million sequence reads of data or more in a 24 hour period from a single machine.

Also included is a computer program, computing device, or analysis platform/system to receive and analyze sequencing data, and output one or more reports that can be transmitted or accessed electronically via a server, an analysis portal, or by e-mail. The computing device or analysis platform can operate according to the algorithms and methods described herein.

### Reaction Mixtures

Also provided herein are reaction mixtures for determining the expression level of a reporter in a sample by sequencing. In some embodiments, the reaction mixture comprises a control nucleic acid provided herein, at least a portion of said biological sample, and one or more enzyme or reagents sufficient to amplify a barcode in a sample, if present.

The control nucleic acid may be any one or more of a single stranded DNA, a double stranded DNA, a single stranded RNA, or a double stranded RNA. In some embodiments, said control nucleic acid is present at a concentration of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 320, 340, 360, 380, 400, 420, 440, 460, 480, or 500 copies per reaction mixture.

In some embodiments, the enzymes or reagents comprise a reverse transcriptase enzyme, dNTPs, a primer pair specific for a barcode or control nucleic acid sequence, a magnesium salt, or combinations thereof.

In some embodiments, the reaction mixture comprises one or more enzymes which can be used to amplify or replicate a control nucleic acid or a barcode nucleic acid. In some embodiments, the enzyme is a reverse transcriptase. Non-limiting examples of reverse-transcriptase enzymes include Avian Myeloblastosis Virus (AMV) Reverse Transcriptase and Moloney Murine Leukemia Virus (M-MuLV, MMLV), and variants thereof.

In some embodiments, the reaction comprises deoxynucleotide triphosphates (dNTPs). In some embodiments, the kit comprises a mixture of each of the dNTPs necessary for amplification of nucleic acids, as well as any other desired nucleic acids (e.g., dATG, dCTP, dTTP, dGTP).

In some embodiments, the reaction mixture comprises a magnesium salt. In some embodiments, the magnesium salt is included in a sufficient quantity to allow the enzymes of the reaction (e.g., the reverse transcriptase enzyme) to function and to amplify targeted nucleic acids. In some embodiments, the magnesium salt is magnesium chloride. In some embodiments, the reaction mixture comprises a concentration of magnesium ions of about 0.1 mM to about 50 mM. In some embodiments, the concentration of magnesium ion is from about 1 mM to about 10 mM.

In some embodiments, the volume of said reaction mixture is from about 10 microliters to about 100 microliters. In some embodiments, the volume of said reaction mixture is from about 20 microliters to about 90 microliters, from about 30 microliters to about 80 microliters, or from about 40 microliters to about 60 microliters. In some embodiments, the volume of said reaction mixture is about 10, 20, 30, 40, 50, 60, 70, 80, 90, or 100 microliters.

### Methods of Quality Control

As described above, a non-limiting example of an assay design by the method described herein may comprise: 1) seeding cells in a well of a well plate; 2) contacting the cells with a test agent allowing time for the test agent to affect reporter activation through a target polypeptide (e.g., barcode expression); 3) lysing the cells to release nucleic acids from the cells, 4) DNase treatment of the lysates to generate associated RNA, 5) reverse transcribing and/or amplifying the reverse transcribed mRNA to create cDNA or amplified cDNA (e.g., RT-PCR); 6) prepare the cDNA for next generation sequencing reaction; and 7) sequencing the prepared cDNA.

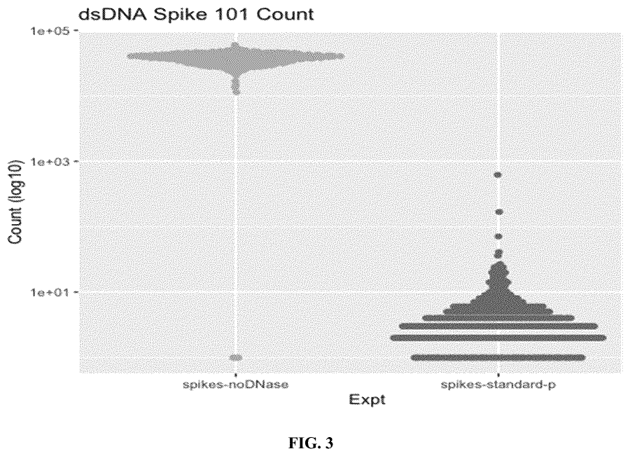

FIG. 3 depicts read counts of a plurality of control nucleic acids added during the DNase step. In this depicted embodiment, the plurality of nucleic acids is a plurality of double stranded DNA spikes. In this example, the plurality of nucleic acids were added during the lysis step. The read counts of the plurality of nucleic acids on the left half of the graph labeled “spikes-noDNase” were taken for an assay in which the DNase step was not performed, while the read counts on the right half of the graph labeled “spikes-standard-p” were taken for an assay where the DNase step was performed. Thus, the read counts on the left half of the graph are large (in between 1e+05 and 1e+04), as the plurality of nucleic acids (e.g., the double stranded DNA spikes) had not been cleaved, and so were still present in solution and able to be identified. By contrast, the read counts on the left are much lower, as many of the plurality of nucleic acids had been cleaved, and so were not still present in solution and were not able to be identified. Thus, with the use of barcodes, the extent of which the DNase step can be evaluated (e.g., lowering the amount of double stranded DNA from around 1e+05 to as low as 0 reads), which can also be evaluated with certain ratios based on the read count (e.g., the DNase inefficiency ratio).

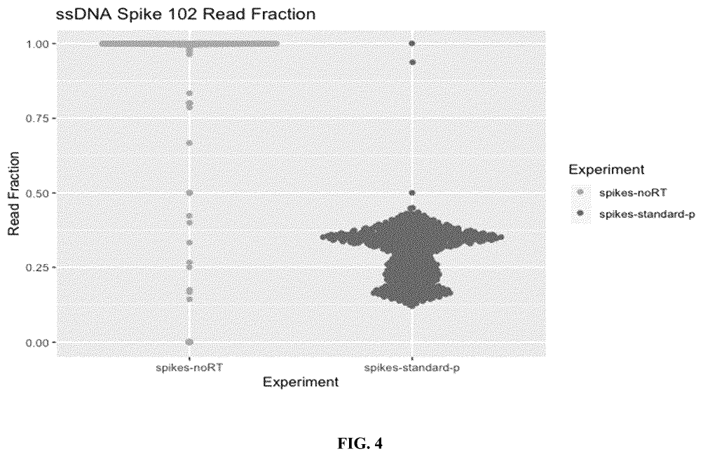

FIG. 4 depicts read fractions of a plurality of control nucleic acids with respect to the total original amount of the plurality of nucleic acids introduced. In this depicted embodiment, the plurality of nucleic acids is a plurality of single stranded DNA. Further, in this depicted embodiment, the plurality of nucleic acids were added just before the RT-PCR step. In this embodiment, the read fractions on the left of the graph, labeled “spikes-noRT”, are read fractions from assays that did not use a reverse transcriptase reagent. Thus, the read fractions tend to be higher, with the majority near 1.0, as the RT-PCR step has not converted any RNA in the samples to DNA, thus the only reads that come through the sequencing reaction are those associated with the ssDNA spike in. The read fractions on the right of the graph, labeled “spikes-standard-p”, are read fractions for assays that perform the RT-PCR step, where the read fractions were determined after the RT-PCR step is performed. Thus, the read fractions show that much of the RNA present in the samples was transcribed into cDNA.

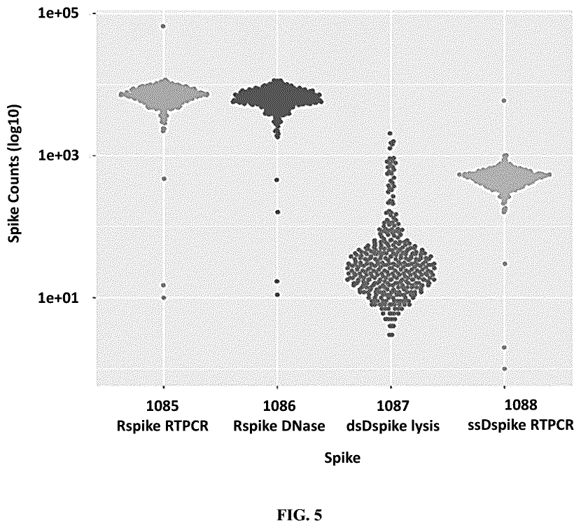

FIG. 5 depicts read counts of various pluralities of nucleic acids introduced in amounts of 10,000 spikes added during one or more steps described above. For example, the read counts labeled “n10185” represent the amount of RNA spikes added during the RT-PCR step, the read counts labeled “n10186” represent the amount of RNA added during the DNase step, the read counts labeled “n10187” represent the amount of double stranded DNA spike that were added during the lysis step, and the read counts labeled “n10188” represent the amount of single stranded DNA during the RT-PCR step. In this depicted embodiment, the n10185 read counts were taken at the beginning of the RT-PCR step, and thus, the read counts are at or just below 10,000 since the reverse transcriptase has not yet been performed. In this depicted embodiment, the n10186 read counts provide information on the RNA preservation during the DNase step, and thus, the read counts are at or just below 10,000, showing that the DNase did not degrade the RNA spikes. In this depicted embodiment, the n10187 read counts provide information on efficiency of the DNase step in degrading DNA, and thus are lower, as many of the double stranded DNA were cleaved during the DNase step. Further, in this depicted embodiment, the n10188 read counts provide information on the efficiency of the RT-PCR step, and thus are lower, as many of the sample RNA were converted to DNA, pushing the overall counts from the added DNA spike lower.

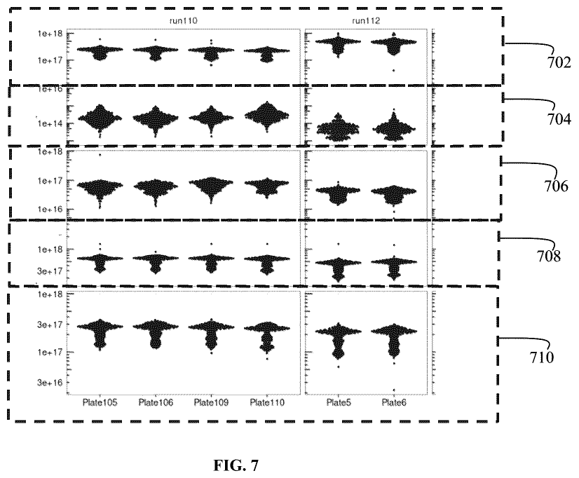

FIG. 7 depicts various read counts of RNA spikes during the DNase step (e.g., read counts 702), DNA spikes during the lysis step (e.g., read counts 704), RNA spikes during the lysis step (e.g., read counts 706), DNA spikes during the RT-PCR step (e.g., read counts 708), and RNA spikes during the RT-PCR step (e.g., read counts 710). The depicted read counts were associated with two different assays (e.g., run110 and run112) and were taken from multiple wells used in each assay (e.g., Plate 105, Plate 106, Plate 109, Plate 110, Plate 5, and Plate 6).

As described above, certain ratios such as the Lysis RNA stability ratio, the DNase inefficiency ratio, the RT efficiency ratio, the RT-PCR RNA stability ratio, and the DNase RNA stability ratio may be calculated from the above results shown in FIGS. 3-7.

### EXAMPLES

The following illustrative examples are representative of embodiments of compositions and methods described herein and are not meant to be limiting in any way.

### Example 1—RNA Based Spike in Control Allows for Rejection of Data from Wells Exhibiting

### Liquid Handling Errors

Specialized process controls that could inform on the performance of our workflow at several critical steps was instituted. These controls comprise various synthetic DNA and RNA molecules spiked into samples in precise quantities at various intermediate reactions. These “spike-ins” then undergo the same downstream processes together with the actual samples. Sequencing readouts of these controls are made at the end of the multiplexed assay, and the results provide key insights into how the process performed and what aspects of the process, if any, experienced problems. These control metrics enabled rapid detection, investigation, and resolution of process issues.

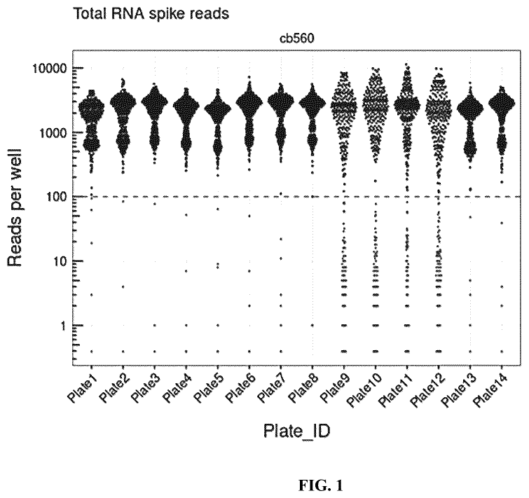

For example, in FIG. 1, an HTS assay was run where cells in wells of a multi-well plate were contacted with a test agent to allow expression (or not) of a barcoded reporter. RNA was harvested reverse transcribed and sequenced using a next generation sequencing assay. The performance of several synthetic RNA spikes provides insight into how multiplexed assay. In the example in FIG. 1, four plates contain a substantial number of wells with a low abundance of these molecules, indicating a process issue at one of our liquid transfer steps. In this figure wells with read counts for the RNA spike in fell below a designated cutoff and were rejected.

In addition to measure the impact of improvements realized by using discrete quality control spike-ins analytics tooling to gauge the quality of data was developed. Most high-throughput biological assays take place in multi-well microtiter plates. Generally, each well of these plates tests a particular experimental condition, namely the effects of a chemical on a cell in the well. Through the process controls mentioned above as well as the behavior of our cell reporters, multiple specific quality control filters can we applied that differentiate poorly performing wells from good ones. Only data coming from high-quality wells are passed into downstream bioinformatics pipelines for analysis. Metrics around how many wells fail QC gates, and why they do, enable monitoring the processes and allow troubleshooting problems in a more focused manner.

### Example 2—RNA Based Spike in Control Allows for Rejection of Data from Wells Exhibiting Liquid Handling Errors

Specialized process controls are determined for a process including the lysis step, DNase step, RT-PCR step, and sequencing step as described above.

A solution with 20 μL of lysis buffer and a plurality of cells are created, and the cells are lysed, DNA spikes (e.g., the second plurality of nucleic acids) and RNA spikes (e.g., the first plurality of nucleic acids) are added to a concentration of 0.5 pM of RNA spikes (e.g., a first plurality of control nucleic acids) and 0.5 pM of DNA spikes (e.g., a second plurality of control nucleic acids).

Eight μL of the lysed solution is transferred to a new well, with 4 μL of buffer added (resulting in a total volume of 12 μL) as well as RNA spike ins (e.g., the fifth plurality of nucleic acids) added to achieve a concentration of 0.5 pM, and the DNase step is performed.

Four μL of the DNased solution is transferred to a new well, and RNA spikes (e.g., the third plurality of nucleic acids) to a total concentration of 0.13 pM and DNA spikes (e.g., the fourth plurality of nucleic acids) to a total concentration of 0.13 pM (resulting in a total volume of 12 μL). RT-PCR is performed. After, the RT PCR step samples are prepared and sequenced on a next generation sequencing machine. Total counts are collected for each of the pluralities after sequencing and the data is used to determine that the assay is carried out to particular tolerances, and data from individual wells are accepted or rejected accordingly. Alternatively, if too many wells fail then the operator can trouble shoot to determine which steps are being performed inefficiently.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

What is claimed is:

1. A method of quality assurance for a high-throughput screening system, the method comprising:
(a) providing a first composition comprising a plurality of cells;
(b) lysing the plurality of cells to obtain a lysed cell composition, wherein the lysed cell composition comprises a first plurality of control nucleic acids, a second plurality of control nucleic acids, or a first plurality and a second plurality of control nucleic acids;
(c) preparing a reverse transcription reaction comprising at least a portion of the lysed cell composition and a third plurality of control nucleic acids, a fourth plurality of control nucleic acids, or a third plurality and a fourth plurality of control nucleic acids;
(d) reverse transcribing the reverse transcription reaction to obtain reverse transcribed nucleic acids and amplifying the reverse transcribed nucleic acids;
(e) preparing a sequencing library comprising the reverse transcribed nucleic acids; and
(f) performing a sequencing reaction on the sequencing library to obtain sequence information for: (i) a plurality of reporters; and (ii) one or more of the first plurality of control nucleic acids, the second plurality of control nucleic acids, the third plurality of control nucleic acids, or the fourth plurality of control nucleic acids.

2. The method of claim 1, further comprising contacting the lysed cell composition with a DNA degrading agent.

3. The method of claim 2, further comprising contacting the lysed cell composition with a fifth plurality of control nucleic acids before contacting the lysed cell composition with the DNA degrading agent.

4. The method of claim 1, further comprising contacting the plurality of cells with a test agent.

5. The method of claim 4, wherein contacting the plurality of cells with the test agent is prior to (b).

6. The method of claim 1, wherein the sequence information for the plurality of reporters provides information on an mRNA expression level for at least one reporter of the plurality of reporters.

7. The method of claim 6, wherein the at least one reporter comprises a barcode sequence.

8. The method of claim 7, wherein the barcode sequence is operably coupled to a promoter and/or an enhancer element.

9. The method of claim 1, comprising performing a sequencing reaction on the sequencing library to obtain sequence information for (i) a plurality of reporters; and (ii) the first plurality of control nucleic acids, the second plurality of control nucleic acids, and the fourth plurality of control nucleic acids.

10. The method of claim 1, wherein the first plurality of control nucleic acids comprises RNA nucleic acids.

11. The method of claim 10, wherein the first plurality of control nucleic acids is added to the plurality of cells prior to lysing the plurality of cells.

12. The method of claim 10, wherein the first plurality of control nucleic acids comprise a first nucleotide sequence, a second nucleotide sequence, a third nucleotide sequence, and/or a fourth nucleotide sequence which are distinguishable from each other.

13. The method of claim 1, further comprising normalizing sequence information for at least one reporter of the plurality of reporters based on the first plurality of control nucleic acids.

14. The method of claim 1, wherein the second plurality of control nucleic acids comprises double stranded DNA nucleic acids.

15. The method of claim 1, wherein the third plurality of control nucleic acids comprises RNA nucleic acids.

16. The method of claim 1, further comprising determining an abundance of the second plurality of control nucleic acids relative to the reverse transcribed nucleic acids.

17. The method of claim 1, wherein the fourth plurality of control nucleic acids comprises single-stranded DNA nucleic acids.

18. The method of claim 1, further comprising determining a control sequence that is not a sequence from the plurality of reporters, the first plurality of control nucleic acids, the second plurality of control nucleic acids, and the fourth plurality of control nucleic acids.

19. The method of claim 1, wherein each of the plurality of reporters comprises a unique barcode sequence and/or each of the plurality of reporters uniquely identify a target molecule.

20. The method of claim 19, wherein the target molecule is a polypeptide expressed from a nucleic acid by the plurality of cells.
